(19) United States
(12) Patent Application Publication
(10) Pub. No.: US 2019/0247560 A1
(43) Pub. Date: Aug. 15, 2019

(54) EXTRACORPOREAL DEVICES AND METHODS OF TREATING COMPLEMENT FACTOR RELATED DISEASES

(71) Applicant: Gambro Lundia AB, Lund (SE)

(72) Inventors: Markus Storr, Filderstadt (DE); Michael Hulko, Bondorf (DE); Bernd Krause, Rangendingen (DE); Werner Beck, Rottenburg (DE); Angelito Bernardo, River Forest, IL (US)

(21) Appl. No.: 15/895,551

(22) Filed: Feb. 13, 2018

(51) Int. Cl. A61M 1/36; A61M 1/34; B01J 20/26

(52) U.S. Cl. CPC A61M 1/362; B01J 20/265; A61M 1/3496

(57) ABSTRACT

The present disclosure relates to devices for the extracorporeal treatment of a patient having a complement factor related disease. The devices are adapted to remove said complement factors from the blood or blood plasma of a patient in need. The disclosure further relates to extracorporeal circuits comprising such devices and methods for the treatment of a patient suffering from a complement factor related disease.

### TECHNICAL FIELD

The present disclosure relates to devices for the extracorporeal treatment of a patient having a complement factor related disease. The devices are adapted to remove said complement factors from the blood or blood plasma of a patient in need. The disclosure further relates to extracorporeal circuits comprising such devices and methods for the treatment of a patient suffering from a complement factor related disease.

### DESCRIPTION OF THE RELATED ART

Therapeutic intervention in the human complement system has long been recognized as a promising strategy for treating a variety of ischemic, inflammatory and autoimmune diseases. Interestingly, the few currently available drugs, such as eculizumab, cover relatively rare diseases and have been developed with the aid of orphan drug regulations. Yet, for many of the more common inflammatory or autoimmune conditions there are no complement drugs available, partly due to the difficulties of developing antibody-based drugs which combine all necessary features of a drug for intravenous in vivo administration, such as, for example, stability, side effects or plasma half-lives. In addition, the costs of the currently available treatments with said drugs are high. Any extension of the current complement-specific therapeutic options would therefore be highly desirable.

Complement factors are components of the complement system which forms a part of the immune system of an individual. The complement system is made up of many distinct plasma proteins that react with one another to destroy pathogens and/or induce a series of inflammatory responses that help to fight infection. Some of the complement proteins are only activated by proteolytic cleavage and could be referred to as inactive precursors. These precursors are widely distributed throughout the body in fluids and tissues without causing any harmful effect. At sites of infection the precursor proteins are activated locally and trigger a series of very efficient inflammatory events, finally resulting in the formation of a membrane attack complex (MAC) which produces holes in the cell membrane of a target cell and causes its destruction (FIG. 1 and FIG. 2); Janeway C A Jr, Travers P, Walport M, et al. Immunobiology: The Immune System in Health and Disease. 5th edition. New York: Garland Science; (2001). The complement system and innate immunity; Horiuchi et al., Inflammation and Regeneration (2016) 36:11.

There are three distinct pathways through which complement activation is triggered by different molecules for their initiation: the lectin pathway (mannan-binding lectin pathway), triggered by the binding of the mannan-binding lectin, a serum protein, to mannose-containing carbohydrates on bacteria or viruses; the classical, antibody-antigen complex pathway, triggered by the binding of C1q to antibody-antigen complexes, which is thus an important link between the effector mechanisms of innate and adaptive immunity; and the alternative pathway, which is initiated when a spontaneously activated complement component binds to the surface of a pathogen (see FIG. 1). Each pathway follows a sequence of reactions to generate a protease type called a C3 convertase, which cleave a very central complement factor, C3, to generate large amounts of C3b, acting as an opsonin and important effector molecule of the complement system, as well as C3a, a peptide mediator of inflammation. C3b also binds to the C3 convertase to form a C5 convertase that produces a very important peptide mediator of inflammation, C5a, and the larger fragment C5b, that contributes to the late events in the complement activation, i.e. the formation of the MAC. In consequence, C3 and C5, due to their key position in complement activation, form two of the most attractive targets for influencing the cascade. However, other complement factors such as 01, C2 and C4 form equally interesting targets due to their role in the system.

The terminal complement components and their function which finally form the MAC are shown in Table I (from Janeway et al. (2001)). A schematic representation of their involvement in MAC formation and the complement pathways is also shown in FIG. 1.

TABLE I
Complement factors forming the membrane attack complex.

| Native | Active | |
|---|---|---|
| protein | component | Function |
| C5 | C5a | Small peptide mediator of inflammation |
| | | (high activity) |
| | C5b | Initiates assembly of the membrane |
| | | attack complex |
| C6 | C6 | Binds C5b, forms acceptor for C7 |
| C7 | C7 | Binds C5b,6, amphiphilic complex inserts |
| | | in lipid bilayer of target cell |
| C8 | C8 | Binds C5b,6,7, initiates C9 polymerization |
| C9 | C9ₙ | Polymerizes to C5b,6,7,8 to form a mem- |
| | | brane-spanning channel, lysing the tar- |
| | | get cell |

In consequence, complement activation happens through a triggered-enzyme cascade. In such a cascade, the activation of a small number of complement proteins at the start of the pathway is amplified by each successive enzymatic or cleavage reaction, resulting in the rapid generation of a very large complement response. In a healthy organism, there are many regulatory mechanisms to prevent uncontrolled complement activation, which is crucial for pathways having the ability to result in such potent inflammatory and destructive effects. It is important in controlling the C1r and C1s activation in the CP, and the MASPs in the LP along with several enzymes in the coagulation system. These mechanisms are well described in detail in Janeway C A Jr, Travers P, Walport M, et al. Immunobiology: The Immune System in Health and Disease. 5th edition. New York: Garland Science; (2001). The complement system and innate immunity; Horiuchi et al., Inflammation and Regeneration (2016) 36:11. It is meanwhile common knowledge that many diseases are connected to a dysregulation of complement activation as described above, often connected to abnormal or uncontrolled, dysregulated activation of the cascade or inadequate performance of complement functions which are often the result of inherited deficiencies or impairment of one or more components of the pathways. Deficiencies of all the soluble complement factors have been described in humans (see, for example, Table 42.2 and pages 593-600 in “Primary Immunodeficiency Diseases: A Molecular & Cellular Approach” by H. D. Ochs et al., Oxford University Press, 2006.)

Deficiencies in the classical pathway can be linked to one or more of the complement factors C1q, C1r, C1s, C4, C2, C1-Inh. Primary deficiency of C1q, C1r, C1s or C4 is closely linked to development of systemic lupus erythematosus (SLE) or rheumatoid arthritis (RA), thought to be due in part to the inability of complement to clear immune complexes and dying cells. Small complexes are cleared from the circulation when they bind to complement receptors on macrophages in the spleen and liver. Without complement, the complexes can grow too large to be easily cleared. The resulting aggregates can activate the alternative pathway, allowing C3 to be deposited into the matrix, with re-solubilized complexes that can be dealt with by the clearance through the liver and spleen. Failing this, these large complexes are no longer soluble, and form deposits in the tissues and become a site of inflammation. Dying cells, if not cleared by non-inflammatory CP activity, may serve as sources of altered self-antigens with the potential for inducing autoantibodies.

C2 deficiency is the most common complement deficiency in Caucasian populations, with frequency estimates between 1 in 10,000 to 1 in 20,000 for homozygous C2-deficient patients. C2 deficiency is found in a slightly higher proportion of SLE patients compared to healthy controls. In primary immunodeficiency, C2 deficiency is found in young children who have recurrent infections, primarily upper respiratory infections with Streptococcus pneumoniae or similar organisms. These children often have frequent ear infections and colds.

Hereditary angioedema (HAE) is a disease caused by deficiency of the CP control protein, C1-Inh. These individuals have recurrent swelling in the extremities, face, lips, larynx or GI tract. The patients suffer from a feeling of fullness but not pain or itching in the affected area except for those with abdominal swellings who often experience acute abdominal pain. The latter two presentations are of the most concern because suffocation can occur if the airways are obstructed, and the acute swelling of the abdominal region produces intense pain often resulting in exploratory surgery. The mechanism for production of the swelling involves not the complement enzymes, but the kinin-generating pathway. It is the production of Bradykinin through this pathway that is responsible for the tissue permeability changes that cause the swelling. Acute treatments include C1 inhibitor (C1-INH, Berinert®) or a replacement therapy; ecallantide, a kallikrein inhibitor; and icatibant, a bradykinin-2 receptor antagonist. Prophylactic treatments include attenuated androgens and C1 inhibitor.

Deficiencies in the alternative pathway (AP) control proteins are also known. Deficiencies of factor H specifically are linked to a wide variety of symptoms. Complete deficiency of factor H leads to uncontrolled activation of the AP and depletion of C3 occurs. This form of factor H deficiency is similar in presentation to the late component deficiencies due to the low or absent levels of C3. The role of this complement control protein is crucial for maintaining health in many tissues. In addition to bacterial infections, deficiency or dysfunction of factor H and the resulting dysregulation of the AP is associated with various forms of kidney disease including atypical Hemolytic Uremic Syndrome (aHUS), as well as age-related macular degeneration (AMD), even though these diseases have also been associated with other complement deficiencies as further described below.

Especially excessive or dysregulated complement activation contributes to many inflammatory, autoimmune and degenerative diseases which have a significant and life-threatening impact on an individual. Some of these diseases are directly caused by genetic changes, mutations or polymorphisms in complement factors, regulators or receptors (Morgan et al. Nature Reviews (2015)14:857-877). For example, diseases connected to primary dysregulation causes the abovementioned aHUS as well as thrombotic thrombocytopenic purpura (TTP), thrombotic microangiopathy, C3 glomerulopathy, transplant rejection and paroxysmal nocturnal haemoglobinuria (PNH). Diseases connected with, for example, C5a, include, but are not limited to, aHUS, PNH, TTP, transplant rejection, sepsis, acute respiratory distress syndrome (ARDS), asthma, systemic lupus erythematosus (SLE), rheumatoid arthritis and chronic obstructive pulmonary disease (COPD), see Marc et al. Scand J Immunol (2010) 71:386-91. Both C3 and C5 (or its cleavage fragment C5a) have been investigated over the last years as they have emerged as leading targets for addressing the diseases connected to complement dysregulation.

Human complement C5 is a beta₁-globulin with a molecular mass of 190 kDa. It is composed of two non-identical polypeptide chains, an alpha-chain of 115 kDA and a beta-chain of 75 kDa. The cleavage of C5 by C5 convertase into C5a and C5b fragments is the first step in the formation of a membrane attack complex. Complement factor C5a directs degranulation of mast cells, chemotaxis of polymorphonuclear leukocytes and contraction of smooth muscle, see again FIG. 2. The assembly of the C5b-9 complex is initiated by the ability of C5b to bind C6 and C7. C5a and its precursor complement factor C5 as well as complement factor C3 (Frémeaux-Bacchi et al., Blood (2008) 112:4948-4952) have especially been investigated in connection with aHUS, a disorder which typically manifests with microangiopathic hemolytic anemia (MAHA), thrombocytopenia and acute renal failure, in severe cases accompanied by fragmented erythrocytes (schistocytosis), see Akesson et al., Therapeutic Apheresis and Dialysis (2017) 21:304-319. The atypical form of aHUS is not associated with Shiga toxin, and the condition is also not thrombotic thrombocytopenic purpura (TTP).

So far, there are basically two approaches to treat aHUS or ameliorate the symptoms of aHUS. Plasmatherapy has been used as one treatment option, wherein plasma exchange seemed to be more efficient than plasma infusions, especially in patients with complete mutant dysfunction of factor H (CFH). However, plasmatherapy does not seem to be effective for treating aHUS with membrane cofactor protein mutation (Loirat et al., Semin Thromb Hemost (2010) 36:673-81). This treatment is otherwise expensive, very complex and associated with other health risks, and burdening for the patient. Anti-complement drugs have gained considerable attention for the treatment of diseases mentioned above. Eculizumab (Soliris; Alexion Pharmaceuticals, Inc., Cheshire, Conn.) is a humanized monoclonal antibody that blocks the cleavage of terminal complement protein C5 into the inflammatory C5a protein and C5b, a precursor of the lytic C5b-9 complex, and currently is the only approved treatment of aHUS. Eculizumab has been demonstrated to be safe and effective in aHUS (Cofiell et al., Blood (2015) 125:3253-3262) and significantly decrease C5a and sC5b-9 levels. Eculizumab is a high-affinity humanized monoclonal anti-05 antibody that blocks terminal complement activity by binding to C5 in a way that blocks the cleavage of C5 into C5a and C5b (WO 1995/029697 A1). Various prior art publications deal with antibodies against C5 and are generally characterized by the ability to inhibit the conversion of the C5 alpha chain to C5a and C5b. The prior art references also describe several diseases which are connected to this complement factor and methods of administering such drug to a patient suffering from a disease which is related to the dysregulation of complement activation (US 2012/0009184 A1; WO 2011/109338 A1; WO 2005/110481 A2; WO 2011/137395 A1; WO 2008/030505 A2; WO 2010/054403 A1; WO 2015/134894 A1; WO 2015/021166 A2; WO 2005/074607 A2; WO 2007/106585 A1; WO 2008/069889 A2; WO 2016/209956 A1; WO 2017/062649 A1; WO 2017/075325 A1; WO 2017/044811 A1; WO 2016/200627 A1; WO 2007/056227 A2; WO 2003/015819 A1; WO 2014/124227 A1; WO 2015/099838 A2, the disclosures of each of which are incorporated herein by reference).

Attempts have long been made to develop inhibitors of complement C5 activation besides eculizumab. These inhibitors target factors upstream of C5, including C5 convertase, complement components C5, C5a, and C5b, and C5a receptor (FIG. 2). Various types of antibodies and compounds such as peptides or non-peptides have actively been developed, and these substances act as inhibitors of complement components C5 and C5a and antagonists of the C5a receptor.

So far, only drugs such as Eculizumab have been provided for the treatment of complement factor related diseases. Anti-complement drugs have the potential to affect each and all of the physiological roles discussed above and it is inevitable that a drug that blocks any of the complement pathways will increase the risk of infections, either non-selectively or for certain groups of organisms. Another major issue is dosing; most complement proteins are abundant in plasma and turn over rapidly, so adequate dosing of an inhibitor can be challenging. It is therefore obvious that large doses of complement-targeting drugs and frequent administration will be needed to block complement at the level of, for example, C3. For example, the Cinryze (a plasma-derived 01 inhibitor; Shire Pharmaceuticals) dose for prophylaxis in hereditary angioedema (HAE) is 1,000 units (100-250 mg) delivered intravenously every 3 days; the eculizumab maintenance dose for adults with PNH is 900 mg every 2 weeks whereas in atypical haemolytic uremic syndrome (aHUS) the maintenance dose is 1,200 mg every 2 weeks. Studies in non-human primates support this—a 5 mg per kg intravenous dose of a FD-specific Fab (lampalizumab, formerly called FCFD4514S, an antigen binding fragment of a humanized monoclonal antibody that binds to complement factor D, Roche/Genentech) inhibited the alternative complement pathway for only 3 hours (Loyet et al. J. Pharmacol. Exp. Ther. (2014) 351:527-537). These relatively high and frequent doses can be contrasted with agents targeting cytokines, which are released de novo in disease and at much lower levels; for example, the tumour necrosis factor (TNF)-specific monoclonal antibody (mAb) adalimumab (Humira; AbbVie) is effective in rheumatoid arthritis at a dose of 40 mg every 2 weeks. The plasma C5 concentration is about 80 mg per liter and the turnover rate is approximately 60 hours; as a consequence, even with high doses, breakthrough activity can occur in some patients treated with eculizumab and monitoring of complement activation in plasma is required. As C5 is not limiting in the complement cascade inhibition at the C5 stage requires near-complete blockade or depletion of C5. Dosing is also complicated because plasma complement factor levels vary widely in individual patients and because many of the factors are acute phase reactants, with synthesis increasing markedly in inflammation, which sometimes causes plasma levels to rise even in the face of increased consumption. Therefore, large amounts of a drug can be needed to effectively inhibit a target protein within complement activation in vivo. Also, owing to their chemical nature, anti-complement agents tend to have short half-lives in vivo. This is not necessarily a limitation, as full coverage can often be achieved through repeated dosing in long-term therapy.

It would be a significant improvement to offer an alternative to the administration of often high doses of anti-complement drugs, most of which are antibodies or peptides, especially for patients which suffer from concomitant ESRD and require hemodialysis either chronically due to an irreversible loss of renal function or as a result of a complement factor related disease. Such alternatives should in addition be cost effective, thereby allowing a more frequent treatment or access as such to a treatment of complement factor related diseases also in less developed countries.

With regard to, for example, aHUS, treatment options for patients with aHUS were limited and often involved plasma infusion or plasma exchange as mentioned herein. In many cases, aHUS patients suffer from renal failure which often becomes chronic. Recently, treatment of aHUS patients with the drug Soliris® became available in the United States of America and in European countries. Despite finally having a useful drug for treatment of aHUS patients, there is still a need to reduce complexity and costs of this treatment, especially in cases where aHUS patients suffer from both aHUS and renal failure and are thus dependent on dialysis in addition to requiring the regular IV administration of Soliris® (Eculizumab).

### SUMMARY

It is an object of the present invention to provide a blood treatment device adapted to remove at least one human complement factor and/or its cleavage fragments from the blood or blood plasma of a patient who suffers from a disease which relates to an acute or chronic, uncontrolled complement activation involving such complement factor and/or any of its cleavage products.

The device is configured to extracorporeally remove said complement factors or fragments thereof from the blood or blood plasma of a patient by immobilizing them on a matrix which is contacted with the said blood or blood plasma of the patient and wherein the matrix comprises a support and a ligand. The support can consist of a membrane, a resin or a non-woven and the ligand is a ligand with high binding affinity towards the targeted human complement factor or fragment thereof. The ligand can, for example, be immobilized on the support covalently, through ionic interaction or complexation.

The ligand can be an antibody or antigen binding fragment thereof which targets the selected complement factor and/or a cleavage fragment thereof. The ligand can also be a peptide aptamer.

The device can generally be designed as a hollow fiber membrane filter or dialyzer wherein the membrane constitutes the support to which a ligand is bound on the lumen side of the hollow fibers which is in contact with blood. The membrane can be a hemodialysis membrane for the treatment of renal failure which is additionally functionalized with said ligands on its lumen side or a plasma separation membrane which is also additionally functionalized with said ligands on its lumen side or, alternatively on the outer side of the hollow fibers and/or its pores.

It is one object of the present invention to provide a hemodialyzer for the purification of blood which can be used for simultaneously treating a patient suffering from renal failure and a disease which is caused by a dysfunction of the complement activation regulation. According to one aspect, the lumen side of the hollow fibers of the hemodialyzer are functionalized with a ligand against a target protein.

According to another aspect, wherein the membranes have a pore size which allows for the passage of a target protein, e.g., a plasma separation membrane, the outer surface and/or the pores of the hollow fiber membrane are functionalized with the ligand. Alternatively, the lumen side of the plasma separation hollow fiber membranes is functionalized with the ligand.

According to yet another aspect, the device can be a hemodialysis filter for the treatment of renal failure wherein the filter further comprises, in at least one of the end caps, a resin, e.g. in sponge form, or a non-woven, which is functionalized with a ligand for immobilizing said complement factor of interest and/or any of its cleavage fragments.

The device can also be an adsorption cartridge comprising a matrix selected from a resin or non-woven material, either of which is functionalized with a ligand configured for binding or immobilizing a target complement factor of interest and/or any of its cleavage fragments (a target protein). Such device can be a member of an extracorporeal circuit for blood treatment, configured to provide hemodialysis, hemodiafiltration, hemofiltration or plasmapheresis. The device can be the sole blood treatment device within the blood circuit or can be located, for example, upstream or downstream of the dialyzer in a hemodialysis, hemodiafiltration or hemofiltration circuit or can alternatively be immediately connected to the dialyzer at the blood inlet or outlet, wherein the device is configured to be perfused with whole blood. The device can also be a member of an extracorporeal plasmapheresis circuit, wherein the device is perfused with blood plasma or components thereof. The device can also be used in therapeutic plasma exchange (TPE) treatment, wherein the plasma is removed from the patient and is then replaced with a substitute, e.g. fresh frozen plasma. According to one aspect of the invention, the device is used to remove a target protein, such as a complement factor, from the substitute plasma of a donor before or during its administration to the patient.

The device can also be a hybrid filter device which combines hollow fiber membranes and a matrix in the filtrate space of the filter (WO 2014/079680 A1), wherein said matrix consists of a resin which is functionalized with a ligand for binding or immobilizing and thus removing a target complement factor and/or at least one cleavage fragment thereof. Such filter can be a member of an extracorporeal circuit configured for performing hemodialysis, hemodiafiltration or hemofiltration, wherein the said filter is located either upstream or downstream of the dialyzer for hemodialysis, hemodiafiltration or hemofiltration, or it can be used as a sole filter device within the said circuit in the absence of such dialyzer. Such device can be used with whole blood.

It is another object of the present invention to provide said extracorporeal circuits which comprise a device configured for the treatment of a complement factor related disease and, optionally, for the concomitant treatment of renal failure.

It is a further object of the present invention to provide devices and extracorporeal circuits as well as methods of treatment which are configured to treat patients suffering from a complement factor related disorder, such as a dysregulation of the complement cascade, wherein the patients are undergoing an acute phase of such disorder which requires immediate and/or prolonged intensive treatment of the condition, such as, for example, in sepsis, and wherein the complement dysregulation is not caused by, for example, an inherited or acquired dysfunction of one or several complement factors. Such treatment can be done over prolonged times such as in CRRT. It is also an object of the present invention to provide devices and extracorporeal circuits as well as methods of treatment which are configured to treat patients suffering from a complement activation disorder, such as a dysregulation of the complement cascade, which is caused, for example, by an inherited defect of at least one complement factor, and wherein the patients are on maintenance treatment, comprising a regular or intermittent treatment with a device according to the invention, optionally together or concomitant with an extracorporeal treatment for renal failure such as hemodialysis or HDF (hemodiafiltration). Optionally such maintenance treatment is done in combination with a standard therapy including the administration of at least one drug for the treatment of the underlying disease.

It is a further object of the present invention to provide a method of treating or ameliorating at least one symptom of a complement factor related disorder in a patient, wherein the method comprises the step of extracorporeally removing at least one complement factor of interest and/or of one or more cleavage fragments thereof from the patient by passing the blood or the blood plasma of the patient over a matrix which is configured to bind or immobilize the said complement factor or one or more of its cleavage fragments, thereby removing it from the blood of the patient. Such removal comprises ex vivo methods wherein, for example, donor blood or donor blood plasma is treated for removing such target complement factor or fragment thereof before further use, e.g. for blood transfusion.

It is another object of the present invention to provide an extracorporeal hemodialysis, hemofiltration or hemodiafiltration circuit for the treatment of end stage kidney disease comprising a device according the invention, wherein the patient does not suffer from any hereditary or otherwise chronic dysregulation of complement activation, but suffers from hemodialysis evoked clinical complications, including chronic inflammation, anemia, and elevated risk of thrombosis and cardiovascular disease, which arise from the contact of artificial filter surfaces with blood constituents, or in other words, from biomaterial surfacetriggered complement activation and subsequent inflammatory and procoagulant reactions. Controlling inflammatory triggers via concomitant removal of complement factor inhibitors, such as, for example, C3 or C5 during hemodialysis treatment (including HDF and HF) could improve the quality of life of an ESRD patient and may beneficially influence the disease state. In any case, the availability of an add-on feature of hemodialysis treatment, either by directly functionalizing the membrane of a hemodialyzer according to the invention or by adopting, upstream or downstream of the dialyzer, a device, such as an adsorber cartridge which is configured to remove the target complement protein, and which can be produced in a cost-efficient manner and easily administered during the standard hemodialysis treatment, would be of particular importance in a market in which cost control is of utmost importance.

It is one object of the present invention to provide for devices, extracorporeal circuits and methods of treating or preventing chronic or acute inflammatory diseases wherein the devices are placed in an extracorporeal blood treatment circuit and are configured to remove a target human complement factor from the blood of a patient.

It is another object of the present invention to provide a method of treating or ameliorating at least one symptom of a human complement factor 5 (C5) and/or C5a and/or C5b related disorder in a patient, wherein the method comprises the step of extracorporeally removing, from the blood or blood plasma of the patient, either C5, C5a or C5b or both C5 and C5a, or both C5 and C5b, or both C5a and C5b, or all of C5, C5a and C5b, by passing the blood or the blood plasma of the patient over a matrix configured to immobilize said components and combinations thereof. Disorders involving, often bedsides other key complement factors such as C3 or C5 and any fragments thereof, include, but are not limited to, aHUS, PNH, ANCA-induced glomerulonephritis (Schreiber et al., JASN (2009): 289-298); chronic obstructive pulmonary disease (COPD) (Marc et al. Scand J Immunol (2010) 71:386-91); respiratory distress syndrome (ARDS); lung injury; rheumatoid arthritis, osteoarthritis, psoriasis, age related macular degeneration (AMD), anti-neutrophil cytoplasmic antibody (ANCA) vasculitis and ischemia-reperfusion injury (Morgan et al., Nat Rev Drug Discov. (2015) 14:857-77); multiple sclerosis, demyelinating peripheral neuropathies, atherosclerosis, multiple organ failure, myocardium damage from reperfusion after ischemia, systemic inflammatory response syndrome (SIRS), multiple organ dysfunction syndrome (MODS), septic shock, toxic shock syndrome, sepsis syndrome, (US 2012/0009184 A1); Degos' disease (WO 2011/109338 A1); an anti-ganglioside or anti glycolipid antibody mediated neuropathy (acute motor axonal neuropathy; acute inflammatory demyelinating polyneuropathy; Bickerstaffs brain stem encephalitis; acute ophthalmoparesis; ataxic GuillainBarre syndrome; pharyngeal cervical-brachial weakness; chronic neuropathy syndromes with anti-glycolipid antibodies; anti-MAG IgM paraproteinemic neuropathy; chronic sensory ataxic neuropathy with anti-disialosyl antibodies; IgM, IgG and IgA paraproteinemic neuropathy; motor neuropathy with anti-GM1 and anti-GM2 antibodies; chronic inflammatory demyelinating neuropathy (CIDP); multifocal motor neuropathy (MMN); and multifocal acquired demyelinating sensory and motor neuropathy (MADSAM)) (WO 2008/030505 A2), complement mediated disorder caused by an infectious agent comprising virus, bacteria, fungi, prion, worm (WO 2016/09483 A2); reducing the likelihood of forming a T cell-mediated allograft vasculopathy lesion in a mammalian transplant recipient (WO 2017/075325 A1); cancer (WO 2017/0246298 A1), tissue graft rejection, ABO incompatibility of transplanted organs with recipient, and hyperacute rejection of transplanted organs.

According to one aspect, medical needs or conditions which can additionally or alternatively be addressed by devices and methods according to the invention involving removing, for example, C3 and/or C5 and/or C5a and/or C5b from the blood of a patient include prolonging survival of allotransplanted cells, tissues or organs and/or the treatment of a patient receiving an ABO-incompatible transplant before and/or after transplantation (WO 2007/103134 A2) and inflammation due to contact of blood with artificial surfaces that may cause complement activation, for example hemodialysis-induced inflammation, inflammation caused by heart-lung machine, for example, in association with vascular surgery as coronary artery bypass grafting or heart valve replacement, plasmapheresis, and the like.

According to one aspect, the present invention provides a method of treating or ameliorating at least one symptom of a human complement factor 5 (C5) and/or C5a related disorders selected from the group of disorders consisting of aHUS, PNH, ANCA-induced glomerulonephritis, chronic obstructive pulmonary disease (COPD), respiratory distress syndrome (ARDS); lung injury; inflammation caused by contact of blood with artificial surfaces such as, for example, hemodialysis-induced inflammation, inflammation caused by heart-lung machine or plasmapheresis, anti-neutrophil cytoplasmic antibody (ANCA) vasculitis, systemic inflammatory response syndrome (SIRS), multiple organ dysfunction syndrome (MODS), septic shock, toxic shock syndrome, sepsis syndrome, and further provides methods for prolonging survival of allotransplanted cells, tissues or organs, treatment of a patient receiving a ABO-incompatible transplant before and/or after transplantation, tissue graft rejection, hyperacute rejection of transplanted organs. According to another aspect, the devices and methods provided herein can be used to treat aHUS, either alone or in combination with other therapies, such as, for example, administration of eculizumab.

It is a further object of the present invention to provide a method of treating or ameliorating at least one symptom of a human complement factor 3 (C3) related disorder in a patient, wherein the method comprises the step of extracorporeally removing, from the blood or blood plasma of the patient, C3 and optionally additionally at least one of its cleavage fragments C3a and/or C3b, by passing the blood or the blood plasma of the patient over a matrix configured to immobilize C3 or C3a or C3b or a combination of C3 and C3a or of C3 and C3b or of C3, C3a and C3b. Disorders involving C3 and/or any of its fragments specifically include, in addition to those mentioned above for C5 and/or its fragments, but are not limited to, aHUS (Frémeaux-Bacchi et al., Blood (2008) 112:4948-4952), PNH, ANCA-induced glomerulonephritis (Schreiber et al., JASN (2009): 289-298). PMC. Web. 2 Feb. 2018.), hemodialysis-induced inflammation (Reis et al. Immunobiology (2014) 220:476482) and C3 glomerulonephritis (Zhang et al., Imunobiology (2015) 220:993-998).

It is one object of the present invention to provide devices for the extracorporeal removal of C3 which comprise compstatin or a compstatin derivative such as APL-2 or Cp40 as a component of their matrix.

It is another object of the present invention to provide a method of treating patients suffering from atypical hemolytic uremic syndrome (aHUS).

### BRIEF DESCRIPTION OF THE DRAWINGS

FIG. 1 schematically describes complement activation pathways (Horiuchi et al. Inflammation and Regeneration (2016) 36:11) and the involvement of complement factors 01, C2, C3, C4, C5 as well as of complement factors C6, C7, C8 and C0 which together form the final MAC, see also Table I. Three pathways, classical, lectin and alternative pathways, are independently activated to form C3. The activation of the cascade through C3 leads to the generation of various fragments including C3a, C3b, C3d, C4a and C5a, which are derived from the precursor complement factors and act as mediators of inflammation by binding to their receptors (see also FIG. 2) on the target cell surface. Another fragment of C5, C5b, together with C6, C7, C8 and multiple copies of C9 lead to the formation of a membrane attack complex which generates lytic pores in the target cell membrane (see also FIG. 2). C3 and C5 have a central role in the cascade and are therefore interesting targets for influencing or silencing complement activation especially in case or dysregulation. Other complement factors involved, such as C4 which also contributes to an amplification within the cascade, and 01 or C2, are equally interesting target proteins. The expression “MASP” is the short version for “mannan-binding lectin-associated serine proteases”.

FIG. 2 shows a schematic representation of the activation of C5 by C5 convertase as shown also in FIG. 1 (Horiuchi et al. Inflammation and Regeneration (2016) 36:11). Cleavage of C5 results in the generation of C5a and C5b. C5a binds to a C5a receptor in the cell wall and mediates several biological responses such as, for example, neutrophil mobilization, histamine release, smooth muscle cell contraction, increased vascular permeability and tissue factor production. C5b initiates the formation of the membrane attack complex which generates lytic pores in the cell membrane and triggers inflammation. This underlines that optionally removing already generated target proteins C5a and/or C5b from the complement activation cascade together with C5 is one of the objects of the invention for providing an efficient and rapid silencing of dysregulated complex activation.

FIG. 3 shows a very schematic representation of an extracorporeal treatment circuit comprising a blood treatment device according to the invention which can be a cartridge or filter comprising a membrane, resin or non-woven based support to which a ligand having affinity for a target protein has been bound. The circuit can be operated in hemoperfusion mode. In cases where the blood treatment device is a hollow fiber membrane filter device the treatment mode can be hemodialysis, hemodiafiltration, hemofiltration or hemoperfusion of the filter with closed dialysate/filtrate ports.

FIGS. 4A and 4B show a very schematic representation of an extracorporeal treatment circuit comprising a blood treatment device according to the invention which can be an adsorption cartridge comprising a resin or non-woven or a filter comprising a membrane, to which a ligand having affinity for a target protein has been bound, respectively. The blood treatment device can be located upstream of a hemodialyzer (pre-dialyzer setting, FIG. 4A) or downstream of a hemodialyzer (post-dialyzer setting, FIG. 4B). The nonfunctionalized hemodialyzer in the circuit can be operated in different treatment modes depending on the medical need, including hemodialysis, hemodiafiltration or hemofiltration mode.

FIG. 5 shows a very schematic representation of an extracorporeal treatment circuit comprising a blood treatment device according to the invention, wherein the blood treatment device is perfused with blood plasma. In the embodiment shown, a plasma separation filter is used to separate blood plasma from whole blood. The plasma filter generates a plasma fraction comprising the target protein by means of pore sizes ranging from 0.03 μm and 2 μm. The plasma is perfused through the blood treatment device which comprises a matrix based on a non-woven, resin or membrane support to which a ligand having an affinity to a target protein has been bound.

FIGS. 6A and 6B schematically depict the covalent coupling of a target protein to an epoxy-activated or an amino support. The support can be a resin, a membrane, including hollow fiber membranes, flat sheet membranes or fiber mats, or a non-woven. FIG. 6A shows the direct coupling of the protein via amino groups of the protein to the support (Example 2). FIG. 6B shows the covalent immobilization of enzymes is based on the use of amino resins. Amino resins can be pre-activated with glutaraldehyde and then used in for covalent immobilization of enzymes. Reaction of an aldehyde group with an amino group of the target proteins is fast and forms a Schiff base (imine), resulting in a stable multipoint covalent binding between enzyme and carrier. The imine double bonds can be further reduced with borohydrides.

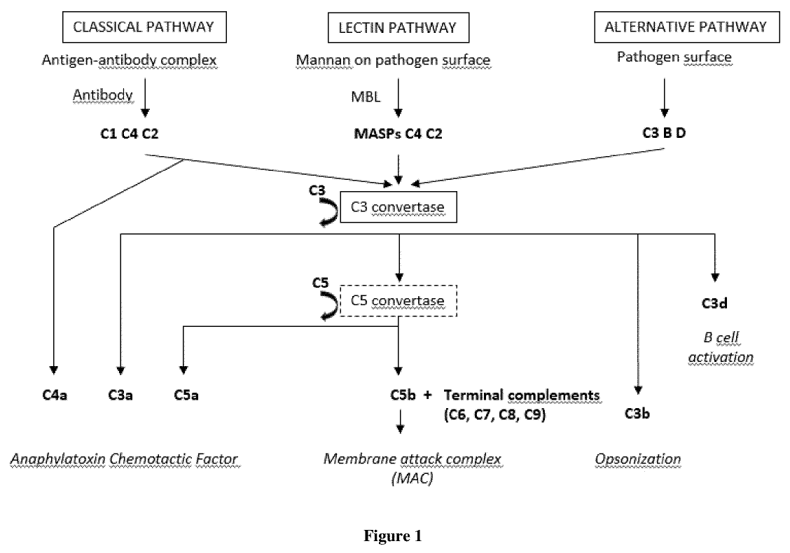

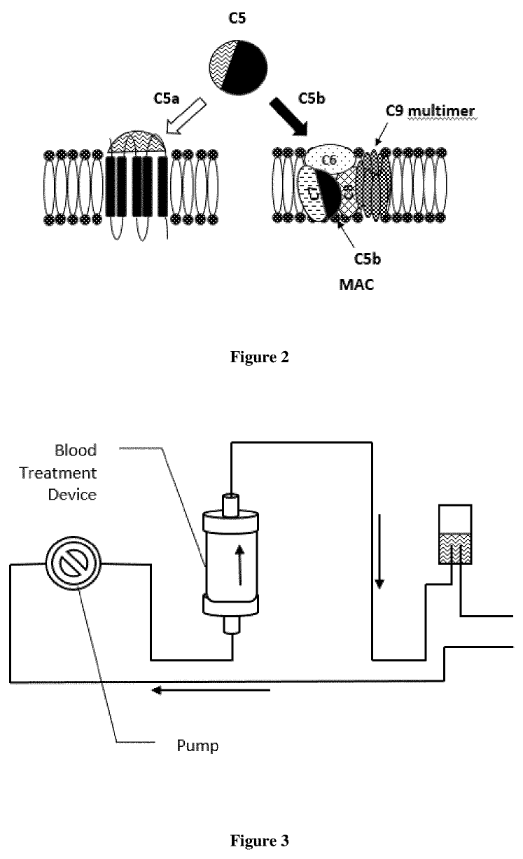

### DETAILED DESCRIPTION

The following numbered embodiments are contemplated and are non-limiting:
1. A blood treatment device adapted to remove at least one human complement factor from the blood or blood plasma of a person in need in an extracorporeal blood circuit, wherein the device comprises a matrix configured to immobilize said complement factor.
2. A blood treatment device according to clause 1, wherein the device comprises a matrix configured to immobilize C5.
3. A blood treatment device according to clause 1, wherein the device comprises a matrix configured to immobilize human complement factor 5a (C5a) and/or human complement factor 5b (C5b).
4. A blood treatment device according to clause 2, wherein the matrix is configured to additionally immobilize human complement factor 5a (C5a) and/or human complement factor 5b (C5b).
5. A blood treatment device according to any of clauses 1 to 4, wherein the blood treatment device is located in an extracorporeal blood circuit through which the blood of the patient passes and which comprises means for transporting blood from the patient's vascular system to the blood treatment device at a defined flow rate and then returning the treated blood back to the patient.
6. A blood treatment device according to any of clauses 1 to 5, wherein the extracorporeal blood circuit in which the blood treatment device is located further comprises a hemodialyzer which is located upstream or downstream of the blood treatment device.
7. A blood treatment device according to any of clauses 1 to 5, wherein the blood treatment device is a hemodialyzer for the hemodialysis of blood which is configured to additionally immobilize C5 and/or C5a and/or C5b.
8. A blood treatment device according to any of clauses 1 to 6, wherein the blood treatment device is an adsorption cartridge which is configured to immobilize C5 and/or C5a and/or C5b and which is perfused with whole blood.
9. A blood treatment device according to any of clauses 1 to 5, wherein the blood treatment device is located in an extracorporeal blood circuit which is configured to separate a blood plasma fraction containing C5, C5a and C5b from the blood, and wherein the blood plasma is passed through the blood treatment device before the treated blood plasma is returned to the patient.
10. A blood treatment device according to clause 9, wherein the extracorporeal blood circuit in which the blood treatment device is located comprises a plasma dialyzer which allows for the separation of said plasma fraction and wherein the blood treatment device is located downstream of the plasma outlet port of the plasma dialyzer.
11. A blood treatment device according to any of clauses 1 to 10, wherein the matrix comprises a support and a ligand which is bound to said support and which is capable of immobilizing at least human complement factor 5.
12. A blood treatment device according to clause 11, wherein the matrix is capable of immobilizing a human complement factor selected from the group consisting of C5, C5a and C5b or combinations thereof.
13. A blood treatment device according to clause 11 or to clause 12, wherein the ligand is an antibody or antigen-binding fragment thereof selected from the group consisting of a humanized antibody, a recombinant antibody, a diabody, a chimerized or chimeric antibody, a monoclonal antibody, a deimmunized antibody, a fully human antibody, a single chain antibody, an Fv fragment, an Fd fragment, a Fab fragment, a Fab′ fragment, and an F(ab′)2 fragment.
14. A blood treatment device according to clause 11 or to clause 12, wherein the ligand is a peptide aptamer.
15. A blood treatment device according to clause 11, wherein the ligand is selected from the group of ligands consisting of eculizumab; LFG316; zimura; ALXN1210; ALXN550; coversin; SOBI002; pexelizumab, MB12/22, MB12/22-RGD, ARC187, ARC1905, SSL7, and OmCI.
16. A blood treatment device according to any of clause 11 to 15, wherein the support is selected from the group of supports consisting of hollow fiber membrane, flat sheet membrane, fiber mat, resin and non-woven.
17. A blood treatment device according to clause 16, wherein the resin is composed of at least one polymer selected from the group consisting of alginate, chitosan, chitin, collagen, carrageenan, gelatin, cellulose, starch, pectin and sepharose; inorganic materials selected from the group consisting of zeolites, ceramics, celite, silica, glass, activated carbon and charcoal; or synthetic polymers selected from the group consisting of polyethylene (PE), polyoxymethylene (POM), polypropylene (PP), polyvinylchloride (PVC), polyvinyl acetate (PVA), polyvinylidene chloride (PVDC), polystyrene (PS), polytetrafluoroethylene (PTFE), polyacrylate, poly(methyl methacrylate) (PMMA), polyacrylamide, polyglycidyl methacrylate (PGMA), acrylonitrile butadiene styrene (ABS), polyacrylonitrile (PAN), polyester, polycarbonate, polyethylene terephthalate (PET), polyamide, polyaramide, polyethylene glycol (PEG), polyvinylpyrrolidone (PVP), polysulfone (PS), polyethersulfone (PES), polyarylethersulfone (PEAS), ethylene vinyl acetate (EVA), ethylene vinyl alcohol (EVOH), polyamide-imide, polyaryletherketone (PAEK), polybutadiene (PBD), polybutylene (PB), polybutylene terephthalate (PBT), polycaprolactone (PCL), polyhydroxyalkanoate, polyether ether ketone (PEEK), polyether ketone ketone (PEKK), polyether imide (PEI), polyimide, polylactic acid (PLA), polymethyl pentene (PMP), poly(p-phenylene ether) (PPE), polyurethane (PU), styrene acrylonitrile (SAN), polybutenoic acid, poly(4-allyl-benzoic acid), poly(glycidyl acrylate), polyglycidyl methacrylate (PGMA), acrylonitrile butadiene styrene (ABS), polydivinylbenzene (PDVB), copolymers of styrene with divinyl-benzene (DVB), poly(allyl glycidyl ether), poly(vinyl glycidyl ether), poly(vinyl glycidyl urethane), polyallylamine, polyvinylamine, copolymers of said polymers and any of these polymers modified by introduction of functional groups.
18. A blood treatment device according to clause 16 and clause 17, wherein the resin is composed of at least one synthetic polymers selected from the group consisting of polymethyl methacrylate) (PMMA), polyglycidyl methacrylate (PGMA), acrylonitrile butadiene styrene (ABS), copolymers of styrene with divinyl-benzene (DVB)polyacrylonitrile (PAN), polyester, polycarbonate, polyethylene terephthalate (PET), polyamide, polyaramide, polyethylene glycol (PEG), polyvinylpyrrolidone (PVP), polysulfone (PS), polyethersulfone (PES), polyarylethersulfone (PEAS), ethylene vinyl acetate (EVA), ethylene vinyl alcohol (EVOH), polyamideimide, polyaryletherketone (PAEK), polybutadiene (PBD), polybutylene (PB), polybutylene terephthalate (PBT), polycaprolactone (PCL), polyhydroxyalkanoate, polyether ether ketone (PEEK), polyether ketone ketone (PEKK), polyether imide (PEI), polyimide, polylactic acid (PLA), polymethyl pentene (PMP), poly(p-phenylene ether) (PPE), polyurethane (PU), styrene acrylonitrile (SAN), polybutenoic acid, poly(4-allyl-benzoic acid), poly(glycidyl acrylate), polyglycidyl methacrylate (PGMA), acrylonitrile butadiene styrene (ABS), polydivinylbenzene (PDVB), poly(allyl glycidyl ether), poly(vinyl glycidyl ether), poly(vinyl glycidyl urethane), polyallylamine, polyvinylamine, copolymers of said polymers and any of these polymers modified by introduction of functional groups.
19. A blood treatment device according to clause 16, wherein the hollow fiber membrane, fiber mat or flat sheet membrane is composed of at least one polysaccharide derivative or synthetic polymer selected from the group consisting of polyacrylate (PA), poly(methyl methacrylate) (PMMA) or polyglycidyl methacrylate (PGMA), polyvinylpyrrolidone (PVP), polysulfone (PS), polyethersulfone (PES), polyarylethersulfone (PAES), combinations of said polymers and any of these polymers modified by introduction of functional groups.
20. A blood treatment device according to clause 16, wherein the non-woven is composed of at least one biopolymer selected from the group consisting of polysaccharide, polylactic acid (PLA), polycaprolactone (PCL) and proteins, or of at least one anorganic material selected from the group consisting of TiO₂, SiO₂ or AlO₂, or from at least one synthetic polymer selected from the group consisting of polypropylene(PP), polyethylene(PE), polyacrylonitrile (PAN), Poly(vinyl alcohol) (PVA), polyamide-imide (PAI), polyurethane (PUR), polyethersulfone (PES), polyacrylic acid (PAA), polyethylene oxide (PEO), polystyrene (PS) and polyvinylidene fluoride (PVDF), combinations of said polymers and any of these polymers modified by introduction of functional groups.
21. An extracorporeal blood circuit through which the blood of a patient passes and which comprises means for transporting blood from the patient's vascular system to a blood treatment device at a defined flow rate and then returning the treated blood back to the patient, wherein the blood treatment device comprises a matrix configured to immobilize at least one human complement factor thereby removing it from the blood of the patient.
22. An extracorporeal blood circuit according to clause 21, wherein the human complement factor is selected from the group consisting of human complement factor 5 (c5), human complement factor 5a (C5a), human complement factor 5b (C5b), and combinations thereof.
23. An extracorporeal blood circuit according to clause 21 or clause 22, wherein the extracorporeal blood circuit further comprises a hemodialyzer for the hemodialysis of blood, and wherein the hemodialyzer is located upstream or downstream of the blood treatment device.
24. An extracorporeal blood circuit according to clause 21 or clause 22, wherein the blood treatment device is a hemodialyzer for the hemodialysis of blood, and wherein the hemodialyzer is configured to additionally immobilize a human complement factor selected from the group consisting of human complement factor 5 (c5), human complement factor 5a (C5a), human complement factor 5b (C5b) and combinations thereof.
25. An extracorporeal blood circuit according to any of clauses 21 to 23, wherein the blood treatment device is an adsorption cartridge and is perfused with whole blood.
26. An extracorporeal blood circuit through which the blood of a patient passes and which is configured to separate the blood plasma from the blood with a plasma filter, wherein the blood plasma is passed through a blood treatment device adapted to remove a human complement factor selected from the group of complement factor 5 (C5), human complement factor 5a (C5a), human complement factor 5b (C5b) and combinations thereof, from the blood plasma of the patient before the treated blood plasma is returned to the patient.
27. An extracorporeal blood circuit according to clause 26, wherein the extracorporeal blood circuit comprises a plasma filter which allows for the separation of plasma and wherein the blood treatment device is located downstream of the plasma outlet port of the plasma dialyzer.
28. An extracorporeal blood circuit according to clause 27, wherein no blood treatment device is located downstream of the plasma filter and wherein the plasma filter is itself adapted to remove human complement factor 5 (C5) from the blood of the patient and wherein the plasma is directly returned to the patient.
29. An extracorporeal blood circuit according to clause 26 and clause 27, wherein the blood treatment device is an adsorption cartridge and is perfused with blood plasma.
30. A method of treating or ameliorating at least one symptom of a human complement factor 5 (C5) related disorder in a patient, wherein the method comprises the step of extracorporeally removing C5 from the patient, wherein the said removing comprises passing the blood of the patient over a device according to any of clauses 1 to 5.
31. A method according to clause 30, wherein the step of removing C5 from the patient comprises passing the blood or the blood plasma of the patient over a matrix configured to immobilize C5.
32. A method according to clause 30 or clause 31, wherein said method further comprises the step of extracorporeally removing in addition at least one cleavage product of C5 consisting of human complement factor 5a (C5a) and human complement factor 5b (C5b) or both.
33. A method according to any of clauses 30 to 32, wherein the human complement factor 5 (C5) related disorder is selected from the group consisting of aHUS, atypical hemolytic uremic syndrome; paroxysmal nocturnal hemoglobinuria; ANCA-induced glomerulonephritis, chronic obstructive pulmonary disease (COPD); rheumatoid arthritis; osteoarthritis; psoriasis; age related macular degeneration (AMD); anti-neutrophil cytoplasmic antibody (ANCA) vasculitis; ischemia-reperfusion injury; multiple sclerosis; demyelinating peripheral neuropathies; atherosclerosis; multiple organ failure; myocardium damage from reperfusion after ischemia, septic shock, toxic shock syndrome, sepsis syndrome; Degos' disease; anti-ganglioside or anti glycolipid antibody mediated neuropathy (acute motor axonal neuropathy; acute inflammatory demyelinating polyneuropathy; Bickerstaffs brain stem encephalitis; acute ophthalmoparesis; ataxic GuillainBarre syndrome; pharyngeal cervical-brachial weakness; chronic neuropathy syndromes with anti-glycolipid antibodies; anti-MAG IgM paraproteinemic neuropathy; chronic sensory ataxic neuropathy with anti-disialosyl antibodies; IgM, IgG and IgA paraproteinemic neuropathy; motor neuropathy with anti-GM1 and anti-GM2 antibodies; chronic inflammatory demyelinating neuropathy (CIDP); multifocal motor neuropathy (MMN); and multifocal acquired demyelinating sensory and motor neuropathy (MADSAM)), hemodialysis-induced inflammation, complement mediated disorder caused by an infectious agent comprising virus, bacteria, fungi, prion, worm.
34. A method according to any of clauses 30 to 33, wherein the human complement factor 5 (C5) related disorder is selected from the group consisting of atypical hemolytic uremic syndrome (aHUS), chronic obstructive pulmonary disease (COPD); anti-neutrophil cytoplasmic antibody (ANCA) vasculitis; multiple organ failure; septic shock; and hemodialysis-induced inflammation.
35. A method according to any of clauses 30 to 34, wherein the serum of the patient shows increased C5b-9 deposition as determined by confocal microscopy and flow cytometry on GPI-AP-deficient cells incubated with aHUS serum compared with a heat-inactivated control or normal serum in an ex vivo assay.
36. A method according to any of clauses 30 to 34, wherein the urine of patients contains elevated levels of at least two aHUS-associated biomarker proteins selected from the group consisting of TNFR1, IL-6, proteolytic fragment Ba of complement component factor B, soluble C5b9 (sC5b9), prothrombin fragment F1+2, d-dimer, thrombomodulin, complement component C5a, β2 microglobulin (132M), clusterin, cystatin C, fatty acid binding protein 1 (FABP-1), soluble CD40 ligand (sCD40L), vascular endothelial cell growth factor (VEGF), chemokine (C-X-C motif) ligand 9, chemokine (C-X-C motif) ligand 10, monocyte chemotactic protein-1, vascular cell adhesion molecule-1, and tissue inhibitor of metalloproteinases-1.
37. A method according to clause 35 or clause 36, wherein the patient in addition received dialysis at least once within the three months immediately prior to treatment with the complement C5 inhibitor; and/or is experiencing a first acute aHUS manifestation.
38. A method according to clause 35 or clause 36, wherein the duration and frequency of the treatment is adapted to achieve a decrease of the levels of the at least two aHUS-associated biomarker proteins and/or a decrease of C5b-9 deposition as determined by confocal microscopy and flow cytometry on GPI-AP-deficient cells incubated with aHUS serum compared to the value prior to treatment.
39. A method according to any of clauses 30 to 38, wherein the method is performed in concurrence with a hemodialysis treatment of the patient suffering from kidney failure.
40. A method according to any of clauses 30 to 38, wherein the method is performed concomitant with the administration of at least one drug for treating a human complement factor 5 related disease.

The present invention is based on the insight that an extracorporeal treatment can be effectively used for the treatment of diseases which are caused by a disorder or dysregulation of complement activation, specifically for diseases wherein at least one complement factor is involved.

As used herein, the terms “complement activation disorder”, “dysregulation of complement activation” and “complement mediated disorder” refer to disorders in which complement activation (e.g., excessive or inappropriate complement activation) is involved, e.g., as a contributing and/or at least partially causative factor. According to one aspect, complement mediated disorders of particular interest are ones in which one or more complement system biomarkers, e.g., one or more genetic markers or biomarkers found in the serum or urine of the patient, is known to be associated with having the disease, such as, for example, in aHUS. According to another aspect, complement mediated disorders also encompass disorders which are not linked to a genetic disposition but involve or are presented by an acute or chronic condition, such as, for example, sepsis or COPD.

The invention includes devices which are configured to be located in an extracorporeal blood circuit through which the blood of a patient passes and which comprises means for transporting blood from the patient's vascular system to a blood treatment device at a defined flow rate and then returning the treated blood back to the patient, and wherein the device is further configured to immobilize at least one of said factors, thereby removing it from the blood of the patient.

The expression “complement component” or “complement factor” as used herein refers to a protein that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, and properdin. Components of the lectin pathway include, e.g., MBL2, MASP-1, and MASP-2. Complement components also include cell-bound receptors for soluble complement components, wherein such receptor mediates one or more biological activities of such soluble complement component following binding of the soluble complement component. Such receptors include, e.g., C5a receptor (C5aR), C5a receptor 2 (C5aR2, often referred to as C5L2) C3a receptor (C3aR), Complement Receptor 1 (CR1), Complement Receptor 2 (CR2), Complement Receptor 3 (CR3, also known as CD45), etc. It will be appreciated that the term “complement factor” is not intended to include those molecules and molecular structures that serve as “triggers” for complement activation, e.g., antigen-antibody complexes, foreign structures found on microbial or artificial surfaces, etc.

Extracorporeal devices and methods for removing target components from the blood of a patient have been described before. For example, WO 2013/020967 A1 discloses the use of a device and matrix for the immobilization and removal of blood group antibodies from a patient. U.S. Pat. No. 8,969,322 B2 described an extracorporeal apheresis procedure for the removal of soluble Flt-1 receptor from the blood of a patient by means of a device comprising dextran sulfate.

Also, as described before, anti-complement drugs are known for the treatment of diseases caused by complement dysregulation and which are directed to a target complement factor such as, for example, C5 or 01. However, despite a wealth of literature on diseases connected to said dysregulation of complement activation and methods to treat at least some of them by antibodies directed against at least one of the complement factors involved, extracorporeal treatment approaches have so far not been described.

The expression “target protein” or “target proteins” as used herein refers to proteins which are components of the complement system and which are involved in complement activation. The expressions “complement factor”, “complement factors” or “complement component(s)” may therefore be used in the same meaning. According to one aspect of the invention, said target protein which is a component of complement activation is a mutated form of the protein, wherein the mutation leads to dysfunction or impaired function or leads to a hyperfunction of the protein, including, for example, increased enzymatic activity, increased binding affinity or increased stability towards being enzymatically altered, such as being cleaved or degraded.

According to one aspect of the present invention, the expression “target protein” or “target proteins” refers to a complement factor (or complement protein) which forms a part of at least one or more of the pathways through which complement activation is triggered, including the lectin pathway (mannan-binding lectin pathway), the classical, antibody-antigen complex pathway, and the alternative pathway. According to another aspect of the invention, the expression “target protein” or “target proteins” refers to a complement factor (or complement protein) which forms a part of the alternative pathway. According to yet another aspect of the invention, the expression “target protein” or “target proteins” refers to a complement factor (or complement protein) which forms a part of the classical pathway. According to another aspect of the invention, the expression “target protein” or “target proteins” refers to a complement factor (or complement protein) which forms a part of the lectin pathway. According to another aspect of the invention, the expression “target protein” or “target proteins” refers to at least one complement factor which is involved in the formation of the membrane attack complex and is selected from the group of factors consisting of C5, C5a, C5b, C6, C7, C8 and C9 or a complex of more than one units of C9.

According to another aspect of the invention, the expression “target protein” or “target proteins” refers to at least one complement factor which is selected from the group of factors consisting of factor B, properdin (factor p), factor Ba, factor Bb, factor D, C1q, C1r, C1s, C4, C2, C2a C1-Inh, C3, C3a, C3b, C4, C5, C5a, C5b, C6, C7, C8, C9 or a complex of more than one units of C9, and C5b-9. According to yet another aspect of the invention, the expression “target protein” or “target proteins” refers to at least one complement factor selected from the group of factors consisting of C3, C3a, C3b, C4, C5, C5a and C5b. According to yet another aspect of the invention, the expression “target protein” or “target proteins” refers to at least one complement factor selected from the group of factors consisting of C3, C3a, C3b, C5, C5a and C5b. According to yet another aspect of the invention, the expression “target protein” or “target proteins” refers to at least one complement factor selected from the group of factors consisting of C3, C3a, C5 and C5a. According to yet another aspect of the present invention, “target protein” or “target proteins” refers to at least one complement factor selected from the group of factors consisting of C5 and C5a. According to yet another aspect of the present invention, “target protein” or “target proteins” refers to at least one complement factor selected from the group of factors consisting of C3 and C3a. According to yet another aspect of the present invention, “target protein” or “target proteins” refers to at least one complement factor selected from the group of factors consisting of C3 and C5.

Accordingly, in one aspect, the invention discloses devices comprising a matrix which is designed for the specific removal from the blood of a patient in an extracorporeal circuit of at least one target protein which is involved in the dysregulation of complement activation. According to another aspect, the invention discloses extracorporeal circuits comprising said devices and describes how such circuits should be configured to effectively treat the blood of the patient in need. According to yet another aspect, the invention provides for a method for reducing the level of at least one target protein in a bodily fluid of a subject, comprising the step of extracorporeally removing the target protein from the patient by passing the blood or the blood plasma of the patient through a device according to the invention. According to one aspect, the device according to the invention comprising an adsorbent, e.g. in the form of beads, has an active surface are, per device, in the range of between 0.5 and 50000 m² when used in whole blood perfusion (hemoperfusion). According to another aspect, the said device according to the invention has an active surface are, per device, in the range of between 0.5 and 50000 m² when used in whole plasma perfusion (therapeutic apheresis). According to yet another aspect, the said devices for hemoperfusion and/or whole plasma perfusion have an active surface area, per device, in the range of between 0.5 and 10000 m².

The expression “blood” as used herein refers to whole blood which contains all components of the blood of an organism, including red cells, white cells, and platelets suspended in plasma. The expression “blood plasma” refers to the fluid, composed of about 92% water, 7% proteins such as albumin, gamma globulin, fibrinogen, complement factors, clotting factors, and 1% mineral salts, sugars, fats, electrolytes, hormones and vitamins which forms part of whole blood but no longer contains red and white cells and platelets. In the context of the present invention, the expression “blood plasma” also refers to specific fractions of the above defined blood plasma in its standard meaning, such as, for example, blood serum.

Various known methods can be used to immobilize a target such as a target protein according to the invention. Such immobilization preferably is specific or selective in that it immobilizes the target protein of interest whereas other proteins and components present in blood or blood plasma or a sample thereof (in vitro) are not immobilized to a significant amount.

According to one embodiment of the invention, one such method is affinity chromatography, also called affinity purification, whereby the target protein is removed from a solution by virtue of its specific binding properties to an immobilized ligand. Affinity chromatography can be defined as a type of liquid chromatography that uses a biologically related agent, that is, an “affinity ligand” or a “ligand”, for selectively retaining a target molecule or to study biological interactions (Hage to al., J. Pharm. Biomed. Anal. (2012), 69, 93-105); Ayyar et al., Methods (2012) 56: 116-129). “Specific binding” generally refers to a physical association between a target molecule (e.g., a polypeptide) or molecular complex and a binding molecule such as an antibody or ligand. The association is typically dependent upon the presence of a particular structural feature of the target such as an antigenic determinant, epitope, binding pocket or cleft, recognized by the binding molecule.

The affinity ligand can consist of a wide variety of binding agents, ranging from a protein or enzyme to an antibody, an antigen, a sequence of DNA or RNA, a biomimetic dye, an enzyme substrate or inhibitor, or a low mass compound (e.g., a drug or hormone). The affinity ligand is immobilized on a support and together with it forms a matrix. It is then used to selectively bind a given target or group of targets within or from a sample, such as, for example, blood or blood plasma. Because of the selective or highly selective nature of many affinity ligands, the matrix can be used to immobilize, bind, isolate, measure, or study specific targets even when they are present in complex biological samples such as blood or blood plasma. In some embodiments, the affinity (as measured by the equilibrium dissociation constant, K_{d}) of two molecules (e.g. between a ligand and a target protein) that exhibit specific binding, is 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, e.g., between 10⁻¹³M and 10⁻⁴ M (or within any range having any two of the afore-mentioned values as endpoints) under the conditions tested, e.g., under physiological conditions regarding, for example, salt concentration, pH, and/or temperature, etc., that reasonably approximate corresponding conditions applied during use according to the invention. Binding affinity can be measured using any of a variety of methods known in the art. For example, assays based on isothermal titration calorimetry or surface plasmon resonance (e.g., Biacore® assays) can be used in certain embodiments. According to one embodiment of the invention, the ligand should have an affinity range of from 10⁻⁶ M to 10⁻¹³ M for the target protein.

The expression “matrix” as used herein thus refers to a material which can be used for affinity chromatography of a target protein according to the invention. Such matrix as used in the context of the present invention comprises a support to which a ligand is bound. The support therefore serves as a carrier for the ligand, even though it has to fulfil other functions as well.

The expression “binding” of or “to bind” (to) a ligand to the support for providing a matrix which can be used in a device according to the invention as used herein refers to a non-covalent or covalent interaction that holds two molecules together. According to one embodiment of the invention, the expression refers to a covalent interaction, i.e. to covalently bound ligands. Non-covalent interactions include, but are not limited to, hydrogen bonding, ionic interactions among charged groups, van der Waals interactions, and hydrophobic interactions among non-polar groups. One or more of these interactions can mediate the binding of two molecules to each other. Binding may otherwise be specific or selective, or unspecific. According to one embodiment of the invention, the expression “binding” of or “to bind” (to) refers to a covalent attachment of the ligand to the support. According to another embodiment of the invention, the expression “binding” of or “to bind” (to) refers to an ionic interaction for the attachment of the ligand to the support.

The expression “ligand” or “ligands” as used herein, generally refers to a molecule which is characterized by its affinity to the target protein. The ligand is further characterized by its specificity for the target protein. It is characterized, according to one embodiment of the invention, by its immobilization feasibility, stability during its use in methods of treating or ameliorating at least one symptom of a human complement factor related disease, and by the retention of target binding capacity after attachment to the matrix over a prolonged time for storage and duration of a treatment of at least 2 hours, preferably of at least 4, at least 8 or at least 12 hours.

According to one embodiment of the invention, ligands represent a group of naturally derived substances such as antibody binding proteins or fragments thereof. In some embodiments of the invention, the ligand is an antibody or an antigen binding fragment thereof, a small molecule, a polypeptide, a polypeptide analog, a peptidomimetic, or an RNA or DNA or peptide aptamer. In some embodiments, the ligand can be one that binds to and immobilizes one or more of complement components C1, C2, C3, C4, C5, C6, C7, C8, C9, Factor D, Factor B, properdin, MBL, MASP-1, MASP-2, or biologically active fragments of any of these components.

According to another embodiment, the ligand can also be a naturally occurring or soluble forms of complement inhibitory compounds such as CR1, LEX-CR1, MCP, DAF, CD59, Factor H, cobra venom factor, FUT-175, compstatin, and K76 COOH. In some embodiments, the ligand can be a complement receptor 2 (CR2)-factor H (FH) molecule comprising: a) a CR2 portion comprising CR2 (e.g., human CR2) or a fragment thereof, and b) a FH portion comprising a FH or a fragment thereof. Exemplary CR2-FH fusion proteins are described and exemplified in, for example, WO 2007/149567 and WO 2011/143637, the disclosures of each of which are incorporated herein by reference. In some embodiments, the ligand comprises a targeting domain such as CR2 or an anti-C3d antibody as described in, for example, WO 2011/163412, the disclosure of which is incorporated herein by reference. Fusions of targeting domains with other complement ligands can be used in the devices and methods described herein as a ligand.

According to another embodiment of the invention, the expression “ligand” or “ligands” represent affinity ligands which are peptides and have been selected based on their binding properties. They are herein referred to as “peptide aptamers”. Such peptide aptamers are small combinatorial proteins that are selected to bind to specific sites on their target molecules (Reverdatto et al., Curr Top Med Chem. 2015; 15(12): 1082-1101). Peptide aptamers consist of short, 5-20 amino acid residues long sequences, sometimes embedded as a loop within a stable protein scaffold. In the context of the present invention, they are immobilized as such or by means of a linker or embedded within a protein scaffold on a support to form the matrix of a device according to the invention in analogy to antibodies or antigen binding fragments thereof. Peptide aptamers which are able to bind to a target protein according to the invention are already known, such as, for example, the cyclic peptide compstatin, which blocks C3 from binding to the convertase (Ricklin et al., Adv Exp Med Biol (2008) 632:283-292) and derivatives thereof, such as, for example, APL-2 (Apellis Pharmaceuticals). Compstatin can therefore also be used as a peptide aptamer ligand according to the invention. Compstatin inhibits the cleavage of native C3 to its active fragments C3a and C3b. As a consequence, the deposition of C3b, the amplification of the alternative pathway and all downstream complement actions are prevented.

The term “antibody” or “antibodies” refers to an an immunoglobulin and encompasses full size antibodies and antibody fragments comprising an antigen binding site. According to one aspect, the antigen is a target protein according to the invention. Antibodies useful in certain embodiments of the invention may originate from or be derived from a mammal, e.g., a human, non-human primate, rodent (e.g., mouse, rat, rabbit), goat, camelid, or from a bird (e.g., chicken), and may be of any of the various antibody isotypes, e.g., the mammalian isotypes: IgG (e.g., of the IgG1, IgG2, IgG3, or IgG4 subclass), IgM, IgA, IgD, and IgE or isotypes that are not found in mammals, e.g., IgY (found in birds) or IgW (found in sharks). An antibody fragment (Fab) may be, for example, a Fab′, F(ab′)2, scFv (single-chain variable), single domain antibody (e.g., a VHH), or other fragment that retains or contains an antigen binding site. See, e.g., Allen, T., Nature Reviews Cancer, Vol. 2, 750-765, 2002, and references therein. Antibodies known in the art as diabodies, minibodies, or nanobodies can be used in various embodiments. Bispecific or multispecific antibodies may be used in various embodiments. The heavy and light chain of IgG immunoglobulins (e.g., rodent or human IgGs) contain four framework regions (FR1 through FR4) separated respectively by three complementarity determining regions (CDR1 through CDR3). The CDRs, particularly the CDR3 regions, especially the heavy chain CDR3, are largely responsible for antibody specificity. An antibody may be a chimeric antibody in which, for example, a variable domain of non-human origin, e.g., of rodent (e.g., murine) or non-human primate origin) is fused to a constant domain of human origin, or a “humanized” antibody in which some or all of the complementarity-determining region (CDR) amino acids that constitute an antigen binding site (sometimes along with one or more framework amino acids or regions) are “grafted” from a rodent antibody (e.g., murine antibody) or phage display antibody to a human antibody, thus retaining the specificity of the rodent or phage display antibody. Thus, humanized antibodies may be recombinant proteins in which only the antibody complementarity-determining regions are of non-human origin. It will be appreciated that the alterations to antibody sequence that are involved in the humanization process are generally carried out through techniques at the nucleic acid level, e.g., standard recombinant nucleic acid techniques. In some embodiments only the specificity determining residues (SDRs), the CDR residues that are most crucial in the antibody-ligand interaction, are grafted. The SDRs is identified, e.g., through use of a database of the three-dimensional structures of the antigen-antibody complexes of known structures or by mutational analysis of the antibody-combining site. In some embodiments an approach is used that involves retention of more CDR residues, namely grafting of so-called “abbreviated” CDRs, the stretches of CDR residues that include all the SDRs. See, e.g., Almagro et al. (2008), Humanization of antibodies. Front Biosci. 13:1619-33 for review of various methods of obtaining humanized antibodies. It should be understood that “originate from or derived from” refers to the original source of the genetic information specifying an antibody sequence or a portion thereof, which may be different from the species in which an antibody is initially synthesized. For example, “human” domains may be generated in rodents (e.g., mice) whose genome incorporates human immunoglobulin genes or may be generated using phage display.

An antibody may be polyclonal or monoclonal, though for purposes of the present invention monoclonal antibodies are generally preferred for generating a device according to the invention. Antibodies may be glycosylated or nonglycosylated. Methods for generating antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture and, e.g., purified from culture medium. Affinity purification may be used, e.g., protein A/G affinity purification and/or affinity purification using the antigen as an affinity reagent. Suitable antibodies can be identified using phage display and related techniques. See, for example, Gary et al. (eds.) “Making and Using Antibodies: A Practical Handbook”. 2^{nd} edition, CRC Press, 2014. Methods for generating antibody fragments are well known. F(ab′)2 fragments can be generated, for example, by using an Immunopure F(ab′)2 Preparation Kit (Pierce) in which the antibodies are digested using immobilized pepsin and purified over an immobilized Protein A column. The digestion conditions may be optimized by one of ordinary skill in the art to obtain a good yield of F(ab′)2. The yield of F(ab′)2 resulting from the digestion can be monitored by standard protein gel electrophoresis. F(ab′) can be obtained by papain digestion of antibodies, or by reducing the S—S bond in the F(ab′)2. Typically, a scFv antibody further comprises a polypeptide linker between the VH and VL domains, although other linkers could be used to connect the domains in certain embodiments.

According to one embodiment of the invention, polyclonal antibodies are used as ligands in a device according to the invention. Polyclonal antibodies are produced from multiple cell lines within the antibody producing organism and as a population can bind a variety of epitopes on a single antigen with a range of binding affinities. Polyclonal antibodies can be produced according to well-known methods against the target by injecting the antigen into an animal as described above. According to one embodiment of the invention, the producing animal is a camelid. This solution of the antigen can contain an enhancing agent called an adjuvant. After this initial injection, blood samples from the animal can be collected after approximately a month (e.g., 3-4 weeks) and tested for the presence of antibodies that are specific for the desired target. Another injection of the antigen (called a ‘booster’) is then made into the animal. The animal's blood is then tested again later (e.g., after 10 days) for the presence of antibodies. This routine of booster administration and testing for antibodies can be repeated several times until the antibody concentrations for the target protein reach the desired level (i.e., as determined by an assay of the blood). At this point, antibody-containing serum can be collected from the animal and stored for later use. The antibodies that are produced upon the first exposure of an animal to a foreign agent are typically IgM class antibodies. After repeated exposure, IgG class antibodies will also be produced. Said IgG class antibodies are generally best suited for IAC applications. Before being used in IAC, the polyclonal antibodies should be further purified by known methods including, but not limited to, ion-exchange chromatography, precipitation with ammonium or dextran sulfate or isolation via a protein A or protein G column. Those antibodies in the antibody mix that do not bind to the target can be removed afterwards, for example, by using an immobilized antigen (target protein) column.

According to another embodiment of the invention, monoclonal antibodies are used as ligands in a device according to the invention. Monoclonal antibodies can be produced by isolating a single antibody-producing cell and combining this cell with a carcinoma or myeloma cell. The resulting hybrid cell line, called a hybridoma, is cultured for long-term antibody production. Because monoclonal antibodies are generated from a single cell line, they bind to a single epitope with identical binding affinities. Individual cultures of hybridomas are examined for the production of specific antibodies and those that make the desired antibody, for example with regard to the desired association equilibrium constant, are cloned to produce a homogenous culture of cells making a monoclonal antibody.

The term “immunoaffinity chromatography” (IAC) is generally used for an affinity chromatography method in which the matrix comprises an antibody or an antigen binding fragment thereof (Moser et al., Bioanalysis (2010) 2:769-790). This selective binding of a target protein to the immobilized antibody is a result of a large variety of noncovalent interactions that can occur between an antibody and an antigen such as the target protein according to the invention and can result in association equilibrium constants in the range of 10⁵-10¹² M⁻¹. In general, a foreign agent that is capable of initiating antibody production in an organism is called an antigen. Due to the generally large size of naturally occurring antigens, antibodies that bind to several different regions of the antigen with a range of binding affinities are often generated. Each individual location on an antigen/target protein that can bind to an antibody is called an epitope. According to one aspect of the present invention, the antibody ligands or antigen binding fragments thereof can bind to any epitope presented by a target protein, provided the association equilibrium constants of the interaction are in the range of 10⁵-10¹⁵ M⁻¹. According to another aspect of the invention, the association equilibrium constants of the interaction are in the range of 10⁶-10¹² M⁻¹, are in the range of 10⁶-10¹⁰ M⁻¹, are in the range of 10⁸-10¹⁰ M⁻¹, or are in the range of 10⁸-10¹² M⁻¹.

According to one specific embodiment of the invention, the antibodies or antigen binding fragments thereof comprise affinity tags for immobilizing them on the support. Affinity tags can be used for purifying the antibodies during their production and/or for immobilizing them on the support of the matrix of the present invention. Affinity tags can be short polypeptide sequences or whole proteins, co-expressed as fusion partners with the target proteins. Apart from facilitating purification and quick immobilization, fusion tags are sometimes also advantageous in increasing the expression and solubility of recombinant proteins. Affinity tags can be used to ensure proper orientation of the antibody, thus, making the functional domains accessible for interaction. They also provide a system for immobilization, quantitation and detection of a target protein and are thus specifically interesting also for analytical purposes, including immunoassays. Different types of affinity tags are well known in the art (Terpe, Appl Microbiol Biotechnol (2003) 60:523-533), wherein polyhistidine or His₆-tags are especially well described and are one option for binding ligands according to the invention to the support material. Affinity tags which can otherwise be used for binding the antibody or an antigen binding fragment thereof to the support can be selected from the group comprising C-myc-tags, FLAG-tags, and Hemagglutinin (HA)-tags.

According to another embodiment of the invention, antibodies or antigen binding fragments thereof can also be immobilized onto supports by using a secondary ligand to adsorb these antibodies. This can be accomplished by using antibodies that have been reacted with biotin or biotinylated, and then adsorbed to a support that contains immobilized avidin or streptavidin. One possible biotinylation technique is to incubate antibodies with N-hydroxysuccinimideD-biotin at pH 9. The noncovalent linkage of biotin to strepavidin or avidin can then be used to immobilize these antibodies. These linkages have association equilibrium constants in the range of 10¹³-10¹⁵ M⁻¹.

According to another embodiment of the invention, the antibodies or antigen binding fragments thereof are covalently attached to the support as further detailed below and/or as described the prior art (Cuatrecasas, J Biol Chem (1970) 245:3059-3065; Nisnevitch et al., J Biochem Biophys Methods (2001) 49:467-480). Covalent coupling generally includes either covalent non-site directed attachment of the antibody or a fragment thereof which is based on utilizing functional groups on either the support and/or the antibody or antibody fragment (Nisnevitch et al., J Biochem Biophys Methods (2001) 49:467-480, Section 2.3). According to another embodiment of the invention the covalent attachment of the antibodies or a fragment thereof is a site-directed attachment of the antibody or antigen binding fragment (Nisnevitch et al., J Biochem Biophys Methods (2001) 49:467-480, Section 2.4; Makaraviciute et al., Biosensors and Bioelectronics (2013) 50:460-471).

Generally, the preparation of a selective immunoaffinity matrix comprising an antibody or antigen binding fragment thereof is well known in the art (Moser et al., Bioanalysis (2010) 2:769-790).

The expression “support” as used herein refers to the portion of the matrix which serves as the “substrate” or “support material” two which the ligands according to the invention are bound. Such support or support material is sometimes also referred to as “adsorption material” or “adsorber” and such expressions shall be encompassed by the expression “support” as used herein. A suitable support according to the present invention should be uniform, hydrophilic, mechanically and chemically stable over the relevant pH range and temperature with no or a negligible leaching of the ligands during use, selective, exhibit minimum non-specific absorption, and should otherwise be blood compatible, i.e. does not induce adverse reactions including the activation of the complement system or other immunological pathways, has good flow characteristics for whole blood and/or blood plasma, and provides a large surface area for ligand attachment.

The support can be a resin, a membrane or a non-woven. The expression “resin” as used herein, refers to an insoluble material which can take the form of translucent gels or gel beads or microporous beads having pores and an opaque appearance, or can take the form of a sponge. Such resins can be natural or bio-polymers, synthetic polymers and inorganic materials. Agarose, dextrose and cellulose beads are commonly employed natural supports. Synthetic polymeric or organic supports are mostly based on acrylamide, polystyrene and polymethacrylate derivatives, whereas, porous silica and glass are some frequently used inorganic supports. Other materials which can be used in accordance with the invention are described below.

According to one embodiment of the invention, the resin is composed of polymers selected from the group consisting of alginate, chitosan, chitin, collagen, carrageenan, gelatin, cellulose, starch, pectin and sepharose; inorganic materials selected from the group consisting of zeolites, ceramics, celite, silica, glass, activated carbon and char-coal; or synthetic polymers selected from the group consisting of polyethylene (PE), polyoxymethylene (POM), polypropylene (PP), polyvinylchloride (PVC), polyvinyl acetate (PVA), polyvinylidene chloride (PVDC), polystyrene (PS), polytetrafluoroethylene (PTFE), polyacrylate (PAA), polymethyl methacrylate (PMMA), polyacrylamide, polyglycidyl methacrylate (PGMA), acrylonitrile butadiene styrene (ABS), polyacrylonitrile (PAN), polyester, polycarbonate, polyethylene terephthalate (PET), polyamide, polyaramide, polyethylene glycol (PEG), polyvinylpyrrolidone (PVP), polysulfone (PS), polyethersulfone (PES), polyarylethersulfone (PEAS), ethylene vinyl acetate (EVA), ethylene vinyl alcohol (EVOH), polyamideimide, polyaryletherketone (PAEK), polybutadiene (PBD), polybutylene (PB), polybutylene terephthalate (PBT), polycaprolactone (PCL), polyhydroxyalkanoate, polyether ether ketone (PEEK), polyether ketone ketone (PEKK), polyether imide (PEI), polyimide, polylactic acid (PLA), polymethyl pentene (PMP), poly(p-phenylene ether) (PPE), polyurethane (PU), styrene acrylonitrile (SAN), polybutenoic acid, poly(4-allyl-benzoic acid), poly(glycidyl acrylate), polyglycidyl methacrylate (PGMA), acrylonitrile butadiene styrene (ABS), polydivinylbenzene (PDVB), poly(allyl glycidyl ether), poly(vinyl glycidyl ether), poly(vinyl glycidyl urethane), polyallylamine, polyvinylamine, copolymers of said polymers and any of these polymers modified by introduction of functional groups. According to one specific embodiment of the invention, the support is selected from the group consisting of styrene divinylbenzene (DVB) and derivatives, polymethyl methacrylate (PMMA) and derivatives, and polyglycidyl methacrylate (PGMA) and derivatives.

As mentioned above, the ligand according to the invention may be covalently bound to the support. The support which forms the basis for the generation of a matrix wherein said ligand can be attached covalently must provide or facilitate chemical activation, thus allowing the chemical coupling of the ligands. Many coupling methods for immobilizing ligands, such as antibodies or fragments thereof, are well known in the art. In general, the activation chemistry should be stable over a wide range of pH, buffer conditions and temperature resulting in negligible leaching of ligands. The coupling method should avoid improper orientation, multisite attachment or steric hindrance of the ligand, which may cause masking of the binding sites and, subsequently, lead to loss of activity. Site-directed attachment and/or spacers can be considered for immobilizing the ligand onto the support. The ligand density per volume of matrix can be optimized to promote target accessibility and binding.

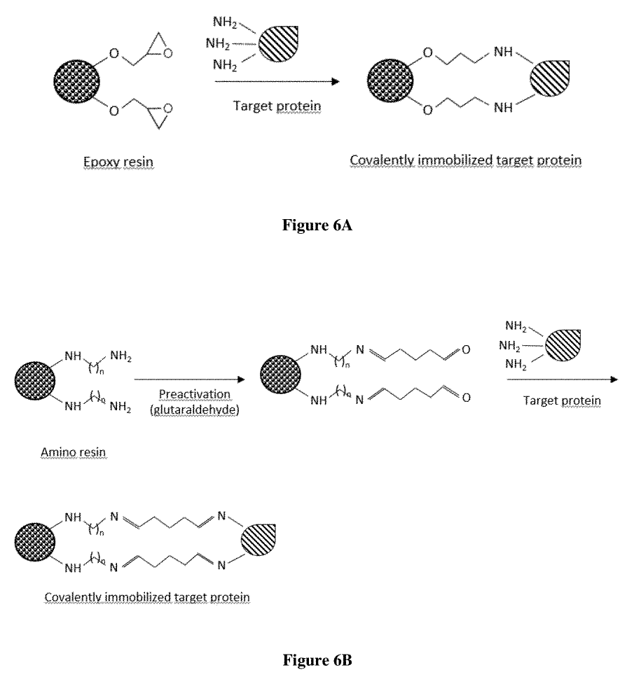

The coupling can be done via common functional groups, including amines, alcohols, carboxylic acids, aldehydes and epoxy groups (FIG. 6A and FIG. 6B). Methods of preparing supports according to the invention are known in the art and are described, for example, in U.S. Pat. No. 8,142,844 B2, US 2015/0111194 A1 and US 2014/0166580 A1. These references also describe spacer groups (or “linker” groups) which can be used in generating the matrix according to the invention.

According to one embodiment of the invention, the ligand is coupled directly or under addition of a spacer via an amine function. In a first step, an amine group is introduced onto the support. Many methods can be used for introducing amine groups to substrates according to the invention. For example, addition of aminated polymers (e.g. aminated polyvinylalcohols) to the polymer solution prior to membrane precipitation, or post-treatment of membranes such as silanization of a membrane containing hydroxyl or/and carboxyl groups using APTMS (3-aminopropyl)trimethoxysilane-tetramethoxysilane), simple adsorption of PEI (poly(ethylene imine)) or other polymers onto the membrane surface, or plasma treatment of the membranes with ammonium or other organic amine vapors can effectively be used to introduce amine groups onto membranes. In a second step, carbodiimide compounds can be used to activate carboxylic groups of proteins for direct conjugation to the primary amines on the membrane surface via amide bonds. The most commonly used carbodiimides are the water-soluble EDC (1-ethyl-3-(-3-dimethylaminopropyl) carbodiimide) for aqueous crosslinking and the water-insoluble DCC (N′, N′-dicyclohexyl carbodiimide) for non-aqueous organic synthesis methods. According to another embodiment of the invention, hydroxyl groups can be introduced to the support. Substrates based on polysaccharides, for example cellulose or cellulose derivatives, already carry OH-groups on the surface. Hydroxy groups can also be introduced to the substrate for example by plasma treatment with oxygen or air. After acylation of the hydroxy group with succinic anhydride the resulting O-succinate can react with amine of the protein with amide bond formation in the presence of carbodiimide or other coupling reagents. According to yet another embodiment of the invention, carboxylic acid groups can be introduced to the support. Carboxylate groups can be introduced on substrates by plasma treatment with carbon dioxide. For protein immobilization carbodiimide/succinimide coupling chemistry can be used for surface attachment via amine group of the ligand. According to yet another embodiment of the invention, carbonyl groups (aldehydes, ketones) can be introduced for the subsequent coupling of a ligand. Aldehydes can be created on polysaccharide-based solid substrates by oxidation of OH-groups using periodic acid. The primary amines of proteins (N-terminus of polypeptides and the side chain of lysines) can react with aldehydes via reductive amination and formation of a Schiff base. The Schiff base formed then hydrolyzes in an aqueous solution and must be reduced to an alkylamine linkage for stabilization. Sodium cyanoborohydride is a mild reducing agent that induces this reaction without also reducing other chemical groups of proteins. According to still another embodiment of the invention, epoxy groups can be introduced to a support. Several pre-activated resins coated with high density epoxy functional groups on the surface are available commercially, see below. The introduction of epoxy groups on membranes is described, for example, in WO 2005/026224 A1. The epoxide group which reacts with nucleophiles in a ring-opening process reacts with primary amines, thiols or hydroxyl groups of proteins to form stable secondary amines, thioesters and ether bonds, respectively. The epoxide groups readily react with thiol groups and require buffered systems close to physiological pH (pH 7.5-8.5). The epoxide groups require high pH conditions (pH 11-12) for reacting with hydroxyl groups and moderate alkaline conditions (pH>9) for reaction with amine groups. In each case, spacers of varying chain length can be introduced between the support and the affinity ligand.

There are several types of supports as mentioned above and below that can be advantageously utilized to couple, for example, antibodies for use in immunoaffinity chromatography. Immunoaffinity supports can be based on materials such as polysaccharide. Suitable polysaccharides are, for example, cellulose, nitrocellulose, chitosan, collagen, starch and cross-linked polysaccharide gels such as agarose, Sephadex or Sepharose. Methods for preparing derivatives of polysaccharide matrices have long been known and are, for example, described in U.S. Pat. No. 4,411,832 or 3,947,352. The supports can also be based on synthetic organic supports. Synthetic polymeric matrices comprise hydrophilic and hydrophobic synthetic polymers and combinations thereof. Synthetic supports comprise supports selected, for example, from the group of supports consisting of polyacrylamide supports or derivatives thereof; polymethacrylate supports or derivatives thereof; polystyrene supports or derivatives thereof; or polyethersulfone supports or derivatives thereof. Otherwise, derivatized silica, glass or azalactone beads can be used in devices according to the invention. Such devices preferably make use of organic supports. The use of beads may be advantageous in the context of the present invention.

According to one embodiment of the invention, the support material should be porous, wherein the pore size is in the range of from 10 to 200 nm. For immunoaffinity applications the pore size has been found to be optimal in the range of from 30 to 200 nm or in the range of 60 to 200 nm. However, other pore sizes may be advantageous as well depending on the coupling chemistry, spacer and ligand used, and also depending on the target protein. If the support is used in the form of beads, the diameter of such beads may vary of a certain range. It may be advantageous to use beads with a diameter in the range of from 50 to 1000 μm. It may be further advantageous to use beads with a diameter in the range of from 60 to 800 μm, 100 to 700 μm, 120 to 800 μm.

According to one aspect of the present invention, the supports carry specific functional groups which are needed for coupling a linker and/or ligand thereto. For example, many functionalized resins are commercially available and known to a person with skill in the art. Pre-activated resin supports which already carry a reactive group for the coupling of a ligand with or without a spacer are available commercially and eliminate many of the steps of chemical activation of the support prior to use mentioned before, i.e. prior to the coupling of a ligand. Such supports are generally resins as defined before, whereas for membrane and/or non-woven supports the step of activation generally has to be performed before coupling. A wide range of coupling chemistries, involving primary amines, sulfhydryls, aldehydes, hydroxyls and carboxylic acids are available in said commercial supports for covalently attaching ligands. Examples for commercially available activated resins are UltraLink Iodoacetyl resin, CarboLink Coupling resin, Profinity™ Epoxide resin, Affi-Gel 10 and 15, Epoxy-activated Sepharose™ 6B, Tresyl chloride-activated agarose, Tosoh Toyopearl® AF Amino or Epoxy 650-M, ChiralVision Immobead™ 350, Resindion ReliZyme™ EXE 135 or SEPABEADS™ and Purolite® Lifetech™ methacrylate polymers functionalized with epoxy groups.

According to one embodiment of the invention, the support used for the coupling of a ligand is epoxy functionalized because epoxy groups form very stable covalent linkages with different protein groups such as, for example, —NH₂ in lysine or nucleophiles (amino, thiol, phenolic) and immobilization can be performed under mild conditions of pH and temperature.

According to another embodiment of the invention, the support takes the form of beads. According to yet another embodiment of the invention, the support is an epoxy-functionalized methacrylate polymer. According to yet another embodiment of the invention, the support is selected from the group of supports consisting of Tosoh Toyopearl® Epoxy 650-M, ChiralVision Immobead™ 350, Resindion ReliZyme™ EXE 135, Resindion SEPABEADS™ and Purolite® Lifetech™. According to one aspect, Purolite® Lifetech™ ECR8209F epoxy methacrylate beads are used which carry an epoxy group as a functional group to which a ligand can be bound. They have a mean pore diameter of between 1200 and 1800 Å and a particle size of between 150 and 300 μm. According to another aspect, Purolite® Lifetech™ ECR8215M epoxy methacrylate beads are used which carry an epoxy group as a functional group to which a ligand can be bound. They have a mean pore diameter of between 600 and 1200 Å and a particle size of between 300 and 710 μm. According to another aspect, Purolite® Lifetech™ ECR8215F epoxy methacrylate beads are used which carry an epoxy group as a functional group to which a ligand can be bound. They have a mean pore diameter of between 1200 and 1800 Å and a particle size of between 150 and 300 μm.

According to another embodiment of the invention, it is also possible to immobilize the ligand non-covalently to the support, for example ionically or by complexation. However, covalent binding is preferred to avoid the risk of leaching of the ligand from the matrix into the blood or blood plasma of the patient.

According to yet another embodiment, the support according to the invention comprises magnetic beads. Magnetic beads are prepared by entrapping magnetite within agarose or other polymeric material, on which the ligand according to the invention is immobilized. Following the interaction of ligand and target protein, under the influence of a magnet, rapid separation can be achieved. The use of magnetic beads is especially indicated in extracorporeal applications which are in vitro applications and wherein the matrix for immobilizing the target proteins is configured for monitoring the presence and/or concentration of the target proteins in a blood or blood plasma sample or in any other in vitro application comprising the target proteins, for screening or other analytical purposes and is not part of the extracorporeal blood circuit. Following the interaction of ligand and target protein, under the influence of a magnet, rapid separation of the target protein can be achieved.

According to another embodiment of the present invention the support is a membrane. Membranes as components of affinity matrices have been used in protein purification, due to their simplicity, ease of handling, reduced surface area and lower diffusion limitations compared to gels, resins and beads. Membranes have been successfully utilized as affinity membranes for the purification of a recombinant antibodies (Sun et al., J. Sep. Sci., 31 (2008), pp. 1201-1206). Affinity membranes are adaptable to be used in various sizes and formats. The membranes can take the physical form of a hollow fiber or, alternatively, of a flat sheet membrane.

According to one embodiment of the invention, the support membrane is a hollow fiber membrane. According to another embodiment of the invention, a multitude of hollow fiber membranes are formed to a bundle of hollow fibers and embedded in a housing, thus forming a filter or filtration device. According to one embodiment, the support comprises a hemodialysis hollow fiber membrane dialyzer, wherein the filter is a hemodialyzer. Such embodiment provides a combination of two functions and can advantageously utilized as a device for remove human complement factor 5 (C5) from the blood or blood plasma of a person in need in an extracorporeal blood circuit, wherein the device comprises a matrix configured to immobilize C5, because the device simultaneously removes a target protein according to the invention and removes uremic toxins, excess ions and water from the blood of the patient who suffers from renal failure. Accordingly, only one device is needed for the treatment of a patient suffering from a human complement factor related disease, specifically a C5 related disease, and renal failure or impairment which is a common consequence of said human complement factor related diseases, and specifically also in aHUS patients. An extracorporeal circuit is accordingly not basically different from a standard extracorporeal circuit for performing hemodialysis in the treatment of renal failure. The treatment of such patients suffering from renal failure and a complement factor related disease is thus significantly simplified and may help to reduce costs for the cumulative treatment of the patients and increase the treatment options for the patients and attending physicians.

The hollow fiber or flat sheet membranes for use as supports in a device according to the invention may be composed of cellulose, cellulose ester (cellulose acetate and cellulose triacetate), poly(methylmethacrylate)(PMMA), polyamide (PA), other nitrogen-containing polymers (polybenzimidazole, polyacrylonitrile (PAN), polyglycidyl methacrylate (PGMA), polyvinylpyrrolidone (PVP), polysulfone (PS), polyethersulfone (PES), polyarylethersulfone (PAES), combinations of said polymers and any of these polymers modified by introduction of functional groups. According to one embodiment of the invention, the membrane supports according to the invention comprise a polymer selected from the group of polymers consisting of poly(methylmethacrylate)(PMMA), polyamide (PA), polyacrylonitrile (PAN), polyvinylpyrrolidone (PVP), polysulfone (PS), polyethersulfone (PES), polyarylethersulfone (PAES), combinations of said polymers and any of these polymers modified by introduction of functional groups. According to another embodiment of the invention, the membrane supports according to the invention comprise a polymer selected from the group of polymers consisting of polyamide (PA), polyacrylonitrile (PAN), polyvinylpyrrolidone (PVP), polysulfone (PS), polyethersulfone (PES), polyarylethersulfone (PAES), combinations of said polymers and any of these polymers modified by introduction of functional groups. According to yet another embodiment of the invention, the membrane supports according to the invention comprise a polymer selected from the group of polymers consisting of polyvinylpyrrolidone (PVP), polysulfone (PS), polyethersulfone (PES), and polyarylethersulfone (PAES), combinations of said polymers and any of these polymers modified by introduction of functional groups.

For performing a coupling reaction for the subsequent binding of a ligand on the membrane surface, a polymer functionalization step is needed known methods can be used such as described in, for example, US 2015/0111194 A1 and US 2014/0166580 A1. For example, a synthetic material made of an alkane chain like, e.g., polyethylene, does not comprise suitable functional groups for coupling a molecule thereto. Therefore, suitable functional groups have to be introduced chemically after polymer synthesis. A possibility for modifying a polymer is the known method of plasma functionalization which allows, by selection of suitable gas plasma, to introduce functional groups into polymers. This method comprises, for example, the use of ammonia plasma, wherein amino functions are formed on the surface of the treated polymer. Hence, treatment of e.g. polyethylene with ammonia plasma leads to a polyethylene matrix bearing a certain amount of amino functions. These amino groups may afterwards be reacted with a suitable functional group of the linker, e.g. a carboxyl group. Alternatively, the matrix polymer can be functionalized by plasma activation to obtain carboxylic groups. A method for functionalizing a hollow fiber membrane in a continuous manner is further described, for example, in US 2007/0296105 A1. In said method the functional groups comprised introduced by the precursor gas may be amino, carboxyl, aldehyde, ester, epoxy, hydroxyl or sulphonic acids groups. Membranes which can be used as supports according to the invention comprise, for example, plasma separation membranes and hemodialysis membranes known in the art, including, but not limited to, well known high-flux membranes, high cut-off membranes or medium cut-off membranes. It is the goal of plasma separation to have the total plasma protein of the blood in the separated plasma fraction, whereas the larger corpuscular components of the blood, like blood cells and cell debris, are retained by the membrane. Further, such a plasma separation membrane should exhibit a high surface porosity and total porosity of the membrane to achieve high filtration performance. It should also be characterized by a hydrophilic, spontaneously wettable membrane structure, low fouling properties for long term stable filtration, and low protein adsorption. Such a plasma separation membrane preferably has smooth surfaces in contact with blood, thus avoiding or minimizing hemolysis during blood processing. The membrane should show constant sieving properties and filtration behavior over the whole treatment period. It should further exhibit high biocompatibility, low or no complement activation and low thrombogenicity. Membranes suitable for plasma separation which can be used for providing a device according to the invention are known in the art and have been described, for example, in EP 1 875 956 A1 or EP 1 875 957 A1. Other membranes which can be modified and used as supports in devices according to the invention, such as high-flux membranes as used, for example, in the Revaclear® dialyzer, have been described in EP 2 113 298 B1. Medium cut-off membranes as used, for example, in the Theranova® dialyzer have been described US 2017/0165616 A1 and high cut-off membranes as used, for example, in the Theralite® dialyzer, have been described in EP 1 572 330 A1.

According to one embodiment of the invention, the device according to the invention comprises hollow fiber membranes selected from a group of hemodialysis hollow fiber membranes prepared from polysulfone, polethersulfone or polyarylethersulfone and polyvinylpyrrolidone.

A hollow fiber membrane which can advantageously be utilized for providing a device according to the invention preferably has an inner diameter in the range of 100 to 500 μm. According to one embodiment of the present invention the hollow fiber membrane has a wall thickness in the range of from 20 to 150 μm. Lower wall thicknesses are disadvantageous due to reduced mechanical properties of the fiber during production and during its use in the device according to the invention itself. Higher wall thicknesses are disadvantageous because they require increased time intervals to perform the phase inversion process resulting in instable process conditions and an instable membrane.

In one embodiment of the invention, wherein the membrane used for providing a device according to the invention is a plasma separation membrane or is otherwise configured to allow the passage of the target protein according the invention to a significant amount with a sieving coefficient of higher than 0.5 and preferably higher than 0.7 or higher than 0.9, the inner layer or lumen of the hollow fibers which generally is the blood contacting layer, is not functionalized and does not carry any ligand. The ligand is instead coupled via a linker to the outer layer of the hollow fibers, and optionally also to at least a portion of the layer connecting the inner layer with the outer layer, i.e. the pores of the membrane. Accordingly, the functionalization with ligands is present only on the outer filtrate layer and optionally on at least a portion of the pore surface structures connecting the outer and inner layer of the membrane. Such configuration can be applied, for example, for the removal of the target proteins from whole blood which due to their size are able to pass from the inner layer to the outer layer, while larger blood proteins remain on the lumen side of the membrane. As blood components including the target proteins are passaging to the outer layer of the membrane they are immobilized on or bound by the specific ligand.

According to another embodiment of the invention, specifically when the membrane support is a hemodialysis membrane as described above, the hollow fiber membranes are additionally or alternatively functionalized with a ligand according to the invention on the lumen side of the hollow fibers where they can directly interact with and bind or immobilize the target protein comprised in the blood or blood plasma which perfuses the lumen of the hollow fiber membrane.

Another aspect of the invention is a blood treatment device comprising a membrane which is functionalized according to the invention with ligand that is configured to bind or immobilize a target protein. Examples of such devices are dialyzers, hemofilters, and ultrafilters. Such devices generally consist of a housing comprising a tubular section with end caps. A bundle of hollow fiber membranes is usually arranged in the casing in a way that a seal is provided between the first flow space formed by the fiber cavities and a second flow space surrounding the membranes on the outside. Examples of such devices are disclosed in EP 0 844 015 A2, EP 0 305 687 A1, and WO 01/60477 A2.

According to another aspect, the device according to the invention can be a filter device as disclosed in WO 2014/07680 A1, which comprises both a bundle of hollow fiber membranes and a resin in the filtrate space of the device, wherein the resin preferably consists of beads. Such device can be configured in a way to serve as a device for removing a target protein according to the present invention by selecting a membrane which allows the passage of at least the relevant target protein through the membrane wall. The resin in the filtrate space of the device serves as the matrix and comprises a resin support, such as disclosed herein or in WO 2014/07680 A1 to which the ligand having an affinity to the target protein is bound by methods disclosed herein or as otherwise known in the art. According to one aspect, the hollow fiber membrane of said device is a plasma separation membrane which allows passage of the blood plasma together with the target proteins contained therein to pass the membrane and interact with the matrix in the filtrate space, thereby allowing the target proteins to be immobilized on the matrix. The cleansed plasma will reenter the hollow fiber membranes within the same device and the blood can return to the patient. Such device can be located in the extracorporeal circuit either upstream or downstream of a hemodialyzer, such as described in WO 2014/079681 A2, or it can be used as a sole hemoperfusion device within the circuit. In another aspect, the ligand can also or exclusively be bound to the plasma separation membrane as described above, for example to outside and/or pores of the membrane. The resin in the filtrate space can, in one aspect, be configured to remove the same or a different target protein. In another aspect, the resin can be configured to remove, specifically or unspecifically, components different from a target protein, such as for example, proteins not related to complement activation, compounds such as endotoxins, for examples in cases where the patient suffers from sepsis or SIRS, or smaller compounds, such as uremic or liver toxins.

According to yet another embodiment of the invention, the support is a non-woven. The expression “non-woven” as used herein refers to a material which is broadly defined as sheet, fabric or web structure bonded together by entangling fiber or filaments (and by perforating films) mechanically, thermally, or chemically but not by weaving or knitting. They form porous structures which can efficiently be used as a support material in devices according to the invention due to their high filtration efficiency, high surface area and high permeability. Non-wovens and processes for their production, comprising melt-blown non-wovens and spunlaid nonwovens, as well as devices containing such non-wovens are known in the art and have been described, for example, in EP 1 922 097 A1, WO 2007/025738 A2 and in Zhao et al., J Mem Sci (2011), 369: 5-12. Non-wovens can be composed of biopolymers selected from the group consisting of polysaccharides, polylactic acids (PLA), polycaprolactone (PCL) and proteins, from anorganic materials selected from the group consisting of TiO₂, SiO₂ or AlO₂, or from synthetic polymers selected from the group consisting of polypropylene(PP), polyethylene(PE), polyacrylonitrile (PAN), Poly(vinyl alcohol) (PVA), polyamide-imide (PAI), polyurethane (PUR), polyethersulfone (PES), polyacrylic acid (PAA), polyethylene oxide (PEO), polystyrene (PS) and polyvinylidene fluoride (PVDF).

Typically, devices according to the invention are designed as cylinders with a cylindrical housing having at least one inlet and at least one outlet for the blood or blood plasma which is treated with it. Where the device is a hemodialyzer which in addition to the removal of at least one target protein serves for the treatment of renal failure in HD, HDF or HF, the device further comprises an inlet and an outlet for dialysis fluid. Device configurations which can be used according to the invention are generally known and are within the scope of this invention.

Ligands, such as antibodies and aptamer binding fragments which bind to a target protein according to the invention, can either be generated by methods known in the art and as described in this text before, or can be selected from a list of known compounds which have been described in the scientific and patent literature. Compounds which have been shown to target a complement factor, include, but are not limited to, LFG316 (anti-05; Novartis International AG, Basel, Switzerland), Zimura (C5 binding aptamer; Ophthotech Corporation, New York, N.Y., United States), CLG561 (anti-properdin; Novartis and Alcon Inc, Hünenberg, Switzerland); APL-2 (C3 inhibitor; Apellis Pharmaceuticals, Crestwood, Ky., United States); TNT003 and TNT009 (True North Therapeutics Inc., CA, United States, target complement fragment C1s); eculizumab (anti-05; Alexion Pharmaceuticals, Inc, Cheshire, Conn., United States); ALXN1210 and ALXN550 (target C5; Alexion Pharmaceuticals, Inc, Cheshire, Conn., United States); lampalizumab (anti-CFD; Genentech, Inc, South San Francisco, Calif., United States, and F. Hoffmann-La Roche AG, Basel, Switzerland); compstatin (e.g. commercially available from Tocris Bioscience, Bio-Techne Ltd. Belgium, Abingdon, UK); Berinert (CSL Behring), Ruconest (Salix Pharmaceuticals, NJ, United States) and Cinryze (Shire Pharmaceuticals, Dublin, Ireland) are C1INH preparations that target C1; and Coversin (a C5-binding protein; Volution Immuno Pharmaceuticals, Geneva, Switzerland); compstatin derivative Cp40 (AMY-101, Amyndas Pharmaceuticals, PA, United States); SOBI002, a C5-blocker that is based on affibody technology (Swedish Orphan Biovitrum AB, Sweden; a small (˜12 kDa) protein, derived using phage display, binds to C5 and prevents cleavage); MB12/22, MB12/22-RGD, ARC187, ARC1905, SSL7, and OmCI (Alexion Pharmaceuticals, Inc, Cheshire, Conn., United States).

According to one embodiment of the invention, at least one compound selected from the list of said known compounds is immobilized on a support according to the invention. According to one embodiment of a device according to the invention, said device comprises at least one compound selected from the list of said known compounds. According to another embodiment of the invention, an extracorporeal blood treatment circuit comprises a device which comprises at least one compound selected from the said list of known compounds. According to yet another embodiment of the invention, a method for treating a disease which is connected to a human complement factor comprises using, within an extracorporeal circuit, a device which comprises at least one compound selected from the said list of known compounds.

According to on embodiment of the invention, the ligand is eculizumab (Soliris, Alexion Pharmaceuticals, Inc.). Eculizumab is an intravenously (IV) administered humanized monoclonal antibody targeting C5, approved for the treatment of two rare genetic deficiencies of complement inhibition, atypical hemolytic uremic syndrome and paroxysmal nocturnal hemoglobinuria. Eculizumab binds to C5, inhibiting its cleavage into C5a and C5b, thereby preventing MAC formation. APL-2 (POT-4/AL-78898A, Apellis Pharmaceuticals) is an intravitreally administered peptide inhibitor of C3. According to another embodiment of the invention, the ligand is lampalizumab (Le et al. J Pharmacol Exp Ther(2015) 355:288-296). Lampalizumab (INN) is an antigen-binding fragment of a humanized monoclonal antibody that binds to complement factor D. According to yet another embodiment of the invention, the ligand is Cp40 (AMY-101, Amyndas Pharmaceuticals, PA, United States) which binds C3 (Zhang et al., Imunobiology (2015) 220:993-998). According to yet another embodiment of the invention, the ligand is SOBI002, a C5-blocker that is based on affibody technology (Swedish Orphan Biovitrum AB, Sweden). According to another embodiment of any of the devices and methods described herein, the ligand is pexelizumab, a C5-binding fragment of anti-05 antibody (Alexion Pharmaceuticals, Inc.). In some embodiments of any of the devices and methods described herein, the inhibitor of complement is selected from the group consisting of MB12/22, MB12/22-RGD, Commercially available anti-05b antibodies are available from a number of vendors including, e.g., Hycult Biotechnology (catalogue number: HM2080; clone 568) and Abcam™ (ab46151 or ab46168).

An exemplary nucleic acid, which encodes an exemplary C5-binding ligand (pexelizumab), is GATATCCAGATGACCCAGTCCCCGTCCTCCCTGTCCGCCTCTGTGGGCGATAGGGTCAC CATCACCTGCGGCGCCAGCGAAAACATCTATGGCGCGCTGAACTGGTATCAACAGAAAC CCGGGAAAGCTCCGAAGCTTCTGATTTACGGTGCGACGAACCTGGCAGATGGAGTCCCT TCTCGCTTCTCTGGATCCGGCTCCGGAACGGATTTCACTCTGACCATCAGCAGTCTGCA GCCTGAAGACTTCGCTACGTATTACTGTCAGAACGTTTTAAATACTCCGTTGACTTTCG GACAGGGTACCAAGGTGGAAATAAAACGTACTGGCGGTGGTGGTTCTGGTGGCGGTGGA TCTGGTGGTGGCGGTTCTCAAGTCCAACTGGTGCAATCCGGCGCCGAGGTCAAGAAGCC AGGGGCCTCAGTCAAAGTGTCCTGTAAAGCTAGCGGCTATATTTTTTCTAATTATTGGA TTCAATGGGTGCGTCAGGCCCCCGGGCAGGGCCTGGAATGGATGGGTGAGATCTTACCG GGCTCTGGTAGCACCGAATATACCGAAAATTTTAAAGACCGTGTTACTATGACGCGTGA CACTTCGACTAGTACAGTATACATGGAGCTCTCCAGCCTGCGATCGGAGGACACGGCCG TCTATTATTGCGCGCGTTATTTTTTTGGTTCTAGCCCGAATTGGTATTTTGATGTTTGG GGTCAAGGAACCCTGGTCACTGTCTCGAGCTGA (SEQ ID NO:1). Possible vector systems for the expression of, for example, C5-binding or C5a-binding or C5b binding polypeptides from nucleic acids (such as plasmid vector systems) are well known in the art and are available for the expression in a number of cells, including in mammalian cells. An antibody, or an antigen-binding fragment thereof, can be expressed in any appropriate host cells. Appropriate host cells include, for example, yeast, bacteria, insect, plant, and mammalian cells, including bacteria such as E. coli, fungi such as Saccharomyces cerevisiae and Pichia pastoris, insect cells such as SF9, mammalian cell lines (e.g., human cell lines), primary cell lines (e.g., primary mammalian cells), Chinese hamster ovary (“CHO”) cells, and a suitable myeloma cell line such as NSO. The expression vectors can be introduced by methods well known in the art into cells in a manner suitable for subsequent expression of the nucleic acid.

According to another embodiment of the invention, antibodies or antigen-binding fragments thereof which are bound to a support for providing a device according to the invention can be selected, depending on the disease or conditions which is sought to be addressed, from the antibodies, fragments and derivatives thereof as disclosed in US 2012/0009184 A1, WO1995029697A1, WO 2007/103134 A2, WO 2011/109338 A1, WO 2011/137395 A; WO 2008/030505 A2; WO 2015/134894 A1; US 2017/0269102 A1, U.S. Pat. No. 9,732,149 B2, WO 2017/044811; WO 2017/0246298 A1; WO 2007/056227 A2; WO 2003/015819 A1; WO 2016/011264 A1, all of which are expressly incorporated herein by reference.

According to one embodiment, antibodies or antigen binding fragments thereof are used which have been generated against complement factor C5 by methods known in the art and which have an association equilibrium constant in the range of 10⁶-10¹⁵ M⁻¹, are in the range of 10⁶-10¹⁵ M⁻¹, are in the range of 10⁶-10¹² M⁻¹, are in the range of 10⁸-10¹² M⁻¹, are in the range of 10⁶-10¹⁰ M⁻¹, are in the range of 10⁶-10⁸ M⁻¹, are in the range of 10⁸-10¹⁰ M⁻¹, or are in the range of 10⁸-10¹² M⁻¹.

According to the invention, a complement component or complement factor such as, for example, C5, is removed from human blood by means of an extracorporeal blood purification wherein a device is located in the extracorporeal circuit which comprises a ligand which has the capability to bind immobilize such complement factor from blood. Extracorporeal blood purification, related and devices and methods are known in the art. The removal capacity of a device according to the invention for the removal of at least one target protein from the blood of a patient is in the range of from 10 mg to 1000 mg of the at least one target protein. According to one aspect, the removal capacity is in the range of from 50 mg to 800 mg of the at least one target protein. According to another aspect the removal capacity is in the range of from 150 mg to 500 mg of the at least one target protein.

According to the invention, the expression “extracorporeal blood purification” refers to the process of removing substances from body fluids through their clearance from flowing blood in a diverted circuit outside the patient's body (extracorporeal). Said substances may include endogenous toxins (i.e., uremic toxins), exogenous poisons (i.e., ethylene glycol or fungal toxin), administered drugs, viruses, bacteria, antibodies and proteins (i.e., IMHA, myasthenia gravis), abnormal cells (i.e., leukemia), and excessive water. Therapeutic procedures include hemodialysis, including intermittent hemodialysis (HD, HDF, HF) and continuous renal replacement therapy (CRRT); hemoperfusion; and therapeutic apheresis.

According to one aspect, blood flow rates in an extracorporeal blood purification circuit are between 20 ml and 700 ml/min. Typical dialysate flow rates in an extracorporeal circuit comprising a hemodialyzer for the treatment of renal failure either in addition to the blood treatment device according to the invention or in cases where the hemodialyzer in addition is configured to immobilize a target protein is in the range of between 0.5 l/h and 800 ml/min.

In hemodialysis, blood is circulated in an extracorporeal circuit and its composition is modified by the mass transfer of solute and water by diffusive and/or convective forces across an interfacing semipermeable membrane. The magnitude and spectrum of the solute transfer is predicated on the nature of the force(s) imposed across the membrane, on the chemical and physical characteristics of the solute, especially also including size, and the structural properties of the membrane. Hemodialysis is a standard treatment for patients suffering from renal failure.

Hemoperfusion is an adsorptive extracorporeal therapy used to manage endogenous and exogenous intoxications that cannot be cleared efficiently by hemodialysis. Adsorption is the principle of molecular attachment of a solute, such a protein, to a material surface (a matrix). In contrast to the physical separation between blood and dialysate that occurs during hemodialysis, during hemoperfusion blood is exposed directly to an adsorbent with the capacity to selectively or non-selectively bind solutes within the blood path.

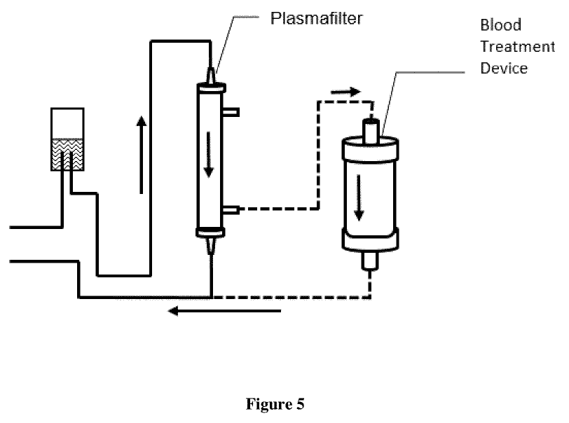

In therapeutic apheresis blood is separated into its component fractions, for example by centrifugation or by means of a plasma membrane or filter, and the fraction containing the solute which shall be removed, generally the plasma fraction, is specifically treated prior to return to the patient. The present invention provides for an apheresis treatment in which plasma (containing the target proteins) is removed from the patient's flowing blood and, after having been contacted with a device or matrix according to the invention is returned to the patient (FIG. 5). Typical plasma flow rates in an extracorporeal circuit wherein the blood treatment device is perfused with blood plasma is in the range of between 7 ml/min and 250 ml/min.

According to one aspect, the extracorporeal blood circuit according to the invention is configured to perform hemodialysis. In this case, the device according to the invention is, for example, a hemodialyzer which additionally has been configured to immobilize a target protein according to the invention. The circuit can be operated in different treatment modes depending on the medical need, including hemodialysis, hemodiafiltration, hemofiltration mode.

According to one aspect, the extracorporeal blood circuit according to the invention is configured to provide continuous renal replacement therapy (CCRT). Continuous renal replacement therapies (CRRT) are slow dialysis treatments that are provided as a continuous 24 hour per day therapy, mostly to critically ill patient in an ICU setting. Like in intermittent HD for chronic renal failure patients, solute removal with CRRT is achieved either by convection (hemofiltration), diffusion (hemodialysis), or a combination of both these methods (hemodiafiltration). This process requires the use of replacement fluid to prevent iatrogenic acidosis and electrolyte depletion as well as excessive fluid removal. CRRT and how to use it is known in the art.

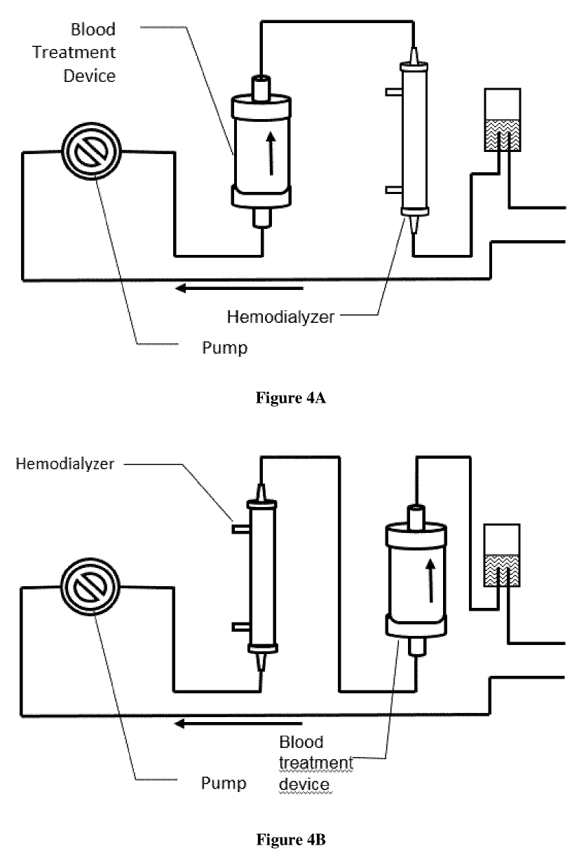

According to another aspect, the extracorporeal blood circuit according to the invention is configured to perform hemoperfusion. Accordingly, the blood treatment device according to the invention is perfused with whole blood and is located within an extracorporeal circuit (FIG. 3). According to one aspect of the invention, the device is a cartridge comprising a membrane, non-woven or resin to which ligands having an affinity for a target protein have been bound. According to one aspect, when the cartridge's matrix comprises a bundle of hollow fiber membrane to which a ligand having affinity to a target protein is bound (a filter device), the treatment mode can be hemoperfusion with closed dialysate/filtrate ports. According to yet another aspect, the cartridge can be located downstream or upstream of a hemodialyzer which is configured to perform hemodialysis on the blood of a patient (FIGS. 4A and 4B) and can be operated in different treatment modes selected from hemodialysis, hemodiafiltration and hemofiltration.

According to one aspect, the blood treatment device is a filter comprising both hollow fibers and a resin in the filtrate space of the filter as described above. The filter can be operated in hemoperfusion mode or, if combined with a hemodialyzer which can be located upstream or downstream of the device according to the invention, the treatment mode can be hemodialysis, hemodiafiltration or hemofiltration.

According to another aspect, the blood treatment device according to the invention is used to ex vivo remove a target protein, such as, for example, C5, C5a, C5b or C3 from donor blood or plasma before it is perfused into a recipient, specifically where the patient suffers or is at risk of suffering from a complement dysregulation.

Devices, extracorporeal circuits and methods according to the invention can be used for the treatment of patients suffering from a disease which is caused by a dysregulation of complement activation. According to one embodiment, devices, extracorporeal circuits and methods according to the invention can be used for the treatment of patients suffering from a disease which is caused by a dysregulation of complement activation. According to one aspect, said dysregulation includes a deficiency or hyperactivity of at least one of the complement factors/target proteins involved.

According to one aspect, devices and methods according to the invention are applicable for diseases selected from the group of diseases including, but not limited to, atypical hemolytic uremic syndrome; paroxysmal nocturnal hemoglobinuria; ANCA-induced glomerulonephritis, chronic obstructive pulmonary disease (COPD); rheumatoid arthritis; osteoarthritis; psoriasis; age related macular degeneration (AMD); anti-neutrophil cytoplasmic antibody (ANCA) vasculitis; ischemia-reperfusion injury; multiple sclerosis; demyelinating peripheral neuropathies; atherosclerosis; multiple organ failure; myocardium damage from reperfusion after ischemia, septic shock, toxic shock syndrome, sepsis syndrome; Degos' disease; anti-ganglioside or anti glycolipid antibody mediated neuropathy (acute motor axonal neuropathy; acute inflammatory demyelinating polyneuropathy; Bickerstaffs brain stem encephalitis; acute ophthalmoparesis; ataxic GuillainBarre syndrome; pharyngeal cervical-brachial weakness; chronic neuropathy syndromes with anti-glycolipid antibodies; anti-MAG IgM paraproteinemic neuropathy; chronic sensory ataxic neuropathy with anti-disialosyl antibodies; IgM, IgG and IgA paraproteinemic neuropathy; motor neuropathy with anti-GM1 and anti-GM2 antibodies; chronic inflammatory demyelinating neuropathy (CIDP); multifocal motor neuropathy (MMN); and multifocal acquired demyelinating sensory and motor neuropathy (MADSAM)), hemodialysis-induced inflammation, complement mediated disorder caused by an infectious agent comprising virus, bacteria, fungi, prion, worm.

According to one aspect of the invention, devices, extracorporeal circuits and methods according to the invention can be used for the treatment of patients suffering from a disease, such as aHUS, which is caused by a dysregulation of the alternative pathway of complement, comprising, but not limited to, at least one mutation in the complement regulatory genes selected from the group of genes consisting of factor H (CFH), membrane cofactor protein (CD46), factor I (CFI), thrombomodulin (THBD); and/or at least one mutation in the activatory genes selected from the group of genes consisting of factor B (CFB) and C3; and/or at least autoantibodies to CFH; see also Rathbone et al. BMJ Open (2013) 3:e003573.

According to another embodiment, devices, extracorporeal circuits and methods according to the invention can be used for supporting ABO-incompatible kidney transplantation and/or for prolonging the life of an allograft, including treating the antibody-mediated rejection of a transplant (AMR), wherein the device is configured to immobilize a target protein on its matrix and wherein the device is located in an extracorporeal blood treatment circuit.

As mentioned before, the diagnosis and monitoring, including monitoring the treatment, of said diseases is not always straightforward but can generally be confirmed by a number of in vitro tests involving selected biomarkers as mentioned below or as described in Cofiell et al. (2015). Diagnosis can be supported by confirming the absence (no identified mutation(s)) or presence (identified mutation) of complement gene mutation(s)/polymorphisms, including but not limited to the genes of CFH, CFI, CD46 (MCP), CFB and C3.

With regard to aHUS, WO 2015/021166 A2 describes methods for evaluating the risk for developing aHUS, diagnosing aHUS, determining whether a subject is experiencing the first acute presentation of aHUS, monitoring progression or abatement of aHUS, and/or monitoring the response to a treatment with a complement inhibitor. According to one embodiment of the invention, the extracorporeal treatment of aHUS according to the invention can be monitored as described in this reference. Specifically, said methods can be used to determine the efficacy of the extracorporeal treatment according to the invention and the frequency of extracorporeal treatments needed to achieve a clinically-meaningful effect on the disease. Said methods may involve comparing the measured concentration or activity of an aHUS biomarker protein (as measured in a biological sample obtained from a subject) to a control sample. In some embodiments, such control sample is obtained from the subject prior to the extracorporeal treatment according to the invention. In another embodiment, the control sample can be (or can be based on), for example, a collection of samples obtained from one or more (two to 40 or more) healthy individuals that have not been received a treatment according to the invention. Said healthy individuals do not have or are not suspected of having (nor are at risk for developing) aHUS.

Accordingly, in one aspect, the method for treating a patient having atypical hemolytic uremic syndrome (aHUS) according to the invention is indicated for patients whose blood or urine has been determined to contain elevated levels of at least two aHUS-associated biomarker proteins selected from the group consisting of TNFR1, IL-6, proteolytic fragment Ba of complement component factor B, soluble C5b9 (sC5b9), prothrombin fragment F1+2, d-dimer, thrombomodulin, complement component C5a, β2 microglobulin (β2m), clusterin, cystatin C, fatty acid binding protein 1 (FABP-1), soluble CD40 ligand (sCD40L), vascular endothelial cell growth factor (VEGF), chemokine (C-X-C motif) ligand 9, chemokine (C-X-C motif) ligand 10, monocyte chemotactic protein-1, vascular cell adhesion molecule-1, and tissue inhibitor of metalloproteinases-1. According to a further aspect, the method is indicated for a patient who in addition the said elevated levels of at least two biomarker proteins received dialysis at least once within the three months immediately prior to treatment with the complement C5 inhibitor; and/or is experiencing a first acute aHUS manifestation. In some embodiments of any of the methods described herein, the concentration of at least two of the group consisting of Ba, sC5b-9, and C5a is measured. According to one specific embodiment, the concentration of one or both of C5a and C5b9 are measured. Complement levels can be detected by antigen assays that quantitate the amount of the protein (CSAG/C5 Complement, Antigen, Serum). Serum C5b-9 levels can be determined using a commercially available enzyme-linked immunosorbent assay kit (Quidel, San Diego, Calif.). Otherwise, the progress of the removal of C5 in the blood or serum of a patient can be measured, for example, by using the “Human Complement C5 ELISA Kit” (Assaypro LLC, MO, United States). According to yet another aspect, the measurement of C5 functional (Wieslab® complement system screen (Euro Diagnostica AB, Malmo, Sweden) and C5 antigen indicate the impact of a treatment according to the invention. Reference values for C5 antigen and C5 functional are 10.6-26.3 mg/dl and 29-53 U/ml, respectively. An option to monitor the removal of C5 and the progress of the treatment can also be done according to Volokhina et al Clinical Immunology (2015) 160:237-243.

According to one aspect of the invention, the method for treating a complement factor related disease comprises the step of extracorporeally removing C5 from the said patient by passing the blood or the blood plasma of the patient over a matrix configured to immobilize C5 with a frequency sufficient to reduce the concentration of the at least two aHUS-associated biomarker proteins compared to the concentration measured in the patient's blood or urine prior to treatment with the complement C5 inhibitor. According to one aspect, a single extracorporeal treatment according to the invention may be performed for 2 to 18 hours, for 2 to 12 hours, for 2 to 8 hours, for 2 to 6 hours, for 3 to 6 hours, for 4 hours. According to another aspect, a single treatment can be repeated when indicated according to the invention. According to another aspect, a single treatment of between 2 and 6 hours can be repeated once, twice, three times or four times per week. In patients suffering from renal failure the treatment according to the invention can be performed concomitant with each hemodialysis treatment.

According to another embodiment of the invention, the diagnosis of a patient for whom the extracorporeal treatment of the invention is applicable and for monitoring the efficacy and progress of the treatment of aHUS according to the invention is done according to Gavriilaki et al. Blood (2015) 125:3637-3646, see also Noris et al. Blood (2014) 124:1715-1726, both of which are incorporated herein by reference, and the serum-based assay that helps to differentiate aHUS from other thrombotic microangiopathies also involving C5 and C5-9. In aHUS patients, increased C5b-9 deposition is evident by confocal microscopy and flow cytometry on glycosyl-phosphatidylinositol-anchored complement regulatory protein (GPI-AP)-deficient cells incubated with aHUS serum compared with heat-inactivated control or normal serum. Normal serum can be, for example, human AB serum (H4522; Sigma-Aldrich, St Louis, Mo.) and can be used in control samples. The method therefore comprises determining increased C5b-9 deposition by confocal microscopy and flow cytometry on GPI-AP-deficient cells incubated with aHUS serum compared with heat-inactivated control or normal serum and, optionally additionally, determining cell viability of biochemically GPI-AP-deficient cells and/or PIGA-deficient cells upon incubation with serum from patients with aHUS (significant increase of nonviable PIGA-deficient TF-1 cells) compared with serum from healthy controls. The method can be applied, for diagnosis, together with a screening for mutations in genes that either regulate or activate the APC, including complement factor H (CFH) and CFH-related proteins, complement factor I, CD46 (membrane cofactor protein), complement factor B, complement component C3, thrombomodulin, plasminogen, diacylglycerolkinase-s (DGKE), and autoantibodies to CFH. Accordingly, it is one object to provide a method of treating aHUS according to the invention in patients with increased C5b-9 deposition on glycosylphosphatidylinositol-anchored complement regulatory protein (GPI-AP)-deficient cells incubated with aHUS serum compared with heat-inactivated control or normal serum. According to a further aspect of the invention, the method comprising the step of extracorporeally removing C5 from the said patient, by passing the blood or the blood plasma of the patient over a matrix configured to immobilize C5 with a frequency sufficient to reduce the C5b-9 deposition on glycosylphosphatidylinositol-anchored complement regulatory protein (GPI-AP)-deficient cells incubated with aHUS serum. Such reduction can be a reduction of from 10% to 100% compared to the heat-inactivated control, TTP, and normal serum. It can be 20% to 80% compared to the heat-inactivated control or normal serum. It can be a reduction of at least 50% compared to the heat-inactivated control, TTP, and normal serum. It can further be a reduction of at least 75% compared to the heat-inactivated control or normal serum. The expression “ameliorating” the condition of a aHUS patient, as used herein, therefore relates to a reduction of C5b-9 deposition on glycosylphosphatidylinositol-anchored complement regulatory protein (GPI-AP)-deficient cells incubated with aHUS serum of at least 20%, 30%, 40%, 50%, 60%, 70%, 80% or 90%, and/or a reduction of the concentration of at least two aHUS-associated biomarker proteins compared to the concentration measured in the patient's blood or urine prior to treatment with the complement C5 inhibitor of at least 20%, 30%, 40%, 50%, 60%, 70%, 80% or 90%. According to one aspect, the treatment of an aHUS patient according to the invention results in a reduction of the at least two biomarkers and/or a reduction of the said C5-9 deposition by 70%, 90% or 100% compared to normal serum. The reduction may be a total reduction after at least one treatment with a device according to the invention or a reduction after several treatments. According to yet another aspect, the treatment will be provided until the patient becomes dialysis independent due to a recovery of renal function. According to yet another aspect, the treatment will be provided until the patients show platelet count normalization.

According to another aspect, the method is provided to patients that additionally show either acute renal impairment or failure or are dependent on chronic renal replacement therapy.

According to another embodiment of the invention, hemolytic assays can be used to identify those patients in need of a treatment according to the invention. The total complement (CH50) assay can be used as a screen for suspected complement deficiencies before investigating into individual complement component assays, such as, for example, C3 or C5. A CH50 in vitro immunoassay for the quantitative determination of total complement activity is commercially available, for example from Wako Diagnostics, VA, United States). Complement levels can then be detected by antigen assays that quantitate the amount of the protein. A deficiency of an individual component of the complement cascade will result in an undetectable total complement level. CH50 tests the capacity of proteins of the classic pathway and membrane attack complex to lyse antibody-coated sheep erythrocytes. The dilution of the serum that lyses 50% of the cells marks the endpoint. The CH50 value is zero in homozygous congenital deficiencies of 01 to C8, and its value is half-normal in C9 deficiency. Also, deficiencies in factors H or I result in a low value due to C3 consumption. The test does not measure deficiencies of the alternative pathway activation proteins. CH50 results are therefore a good indicator for the progress of the treatment according to the invention (Andreguetto et al. (2015) Abstracts/Molecular Immunology 67:119-1120, 003). According to another embodiment of the invention, the alternative hemolytic complement activity (AH50) can be used to measures alternative pathway function that requires the presence of adequate factor B, factor D, and properdin, thus being an option to identify those patients in need of a treatment according to the invention.

According to one embodiment of the invention, the diagnostic and monitoring methods described herein are used to monitor the subject during therapy or to determine effective therapeutic dosages or to determine the number and frequency of treatments needed.

According to yet another embodiment of the invention, patients subjected to a method of treatment according to the invention are vaccinated, before or during receiving said treatment, against encapsulated bacteria, including meningococci, pneumococci, and Haemophilus influenza, in order to eliminate the potential risk for infections during treatment.

According to another aspect, the method of treating a complement factor related disease may be performed concomitant with or in addition to the administration, intravenous, oral or otherwise, of drugs for treating a complement activation dysregulation related disease. For example, a method of treatment according to the invention can be performed in addition to the administration to a drug which blocks a complement component and/or cleavage thereof, especially in cases where the clinical effect of the said drug is not fully satisfactory. According to one aspect, a method of treating aHUS extracorporeally by removing C5 and/or C5a and/or C5b from the blood of a patient, especially of a patient being dialysis dependent, can be performed in addition to the administration of eculizumab (Soliris®). According to another aspect, said extracorporeal treatment according to the invention is performed together with hemodialysis of the patient being treated for renal failure which may be acute or chronic.

According to yet another embodiment of the invention, the devices according to the invention may be regenerated in between treatments.

### EXAMPLES

### Example 1: Preparation of a Matrix Comprising an Epoxy Functionalized Resin

First, the resin is equilibrated. The resin is washed with immobilization buffer and filtered. A resin/buffer ratio of 1/1 (w/v) is preferable. The immobilization buffer is chosen to be compatible with a recombinant anti-human C5 antibody manufactured by BAC B.V. (Naarden, Netherlands) and its stability. The process is repeated for 2-4 times. The antibody solution is prepared by dissolving the native antibody in immobilization buffer. For example, 100-200 mg antibody can be loaded per gram of wet resin. Protein concentration can be determined by using standard protein content assays. The antibody is dissolved in a sufficient amount of buffer to obtain a ratio resin/buffer of 1/4 (w/v). This ratio can be optimized depending on the antibody used (range can vary from 1/1-1/4). Immobilization starts with the transfer of the immobilization buffer containing the antibody into the immobilization vessel. The epoxy-functionalized resin, for example the Purolite® Lifetech™ resin described herein, is then added. The slurry is gently mixed at 70-80 rpm for 18 h and afterwards left without mixing for another 20 h. Magnetic stirring during protein immobilization should be avoided as this can damage the beads. Immobilization can be performed at temperatures of 20° C.-30° C., depending on the protein stability. Immobilizations should not be performed at high temperatures as this can cause degradation of the epoxy rings (hydrolysis) and facilitate microbial growth. Finally, the liquid phase is filtered of and collected. The protein content in the liquid is determined and the immobilization yield evaluated. The resin is then washed with washing buffer. The process is repeated for 2-4 times under gentle stirring or in column wash. An additional washing step using a 0.5 M NaCl containing buffer for complete desorption of non-covalently bound proteins can be done. Excess water is removed by filtration. The immobilized antibody can then be characterized in terms of moisture content and specific binding activity.

### Example 2: Preparation of a Matrix Comprising an Epoxy-Functionalized Resin

First, the resin is equilibrated. The resin is washed with immobilization buffer and filtered. A resin/buffer ratio of 1/1 (w/v) is preferable. The immobilization buffer is chosen to be compatible with the antibody and its stability. In a second step 2% glutaraldehyde buffer is prepared starting from a solution of 25% (w/v) glutaraldehyde. A 2% glutaraldehyde (v/v) solution is prepared using the immobilization buffer. In a third step, the amino resin is activated by adding the 2% glutaraldehyde buffer prepared in step 2 to the resin. The optimal volume of 2% glutaraldehyde buffer should be in the range of resin/buffer ratio of 1/4 (w/v). The slurry is left to mix for 60 min at 20° C.-25° C. The beads are then filtered and washed with immobilization buffer using a resin/buffer ratio of 1/4 (w/v). It should be avoided to store pre-activated resin for a period longer than 48 h. Beads are then ready for the immobilization step. In a fourth step the protein (antibody) solution is prepared. To that end, the protein is dissolved in immobilization buffer. For example, between 1 mg and 100 mg antibody can be loaded per gram of wet resin. The protein concentration can be determined by using standard protein content assays.

The protein is dissolved in buffer to obtain a ratio resin/buffer of 1/4 (w/v). Optimization of this ratio can be pursued in further trials (range can vary from 1/1-1/4). In a fifth step, the protein is immobilized. The immobilization buffer is transferred into the immobilization vessel and add the pre-activated amino resin (e.g. from Purolite®, Lifetech™) as prepared in step 3. The slurry is gently mixed for 18 h at 70-80 rpm. Magnetic stirring should be avoided during immobilization as this can damage the beads. The immobilization can be performed at 20° C.-30° C. accordingly to antibody stability. The immobilization should not be performed at high temperatures since this might cause side reactions of the aldehyde groups on the resin formed during step 3. Finally, the liquid phase is filtered of and collected. The protein content in the liquid is determined and the immobilization yield evaluated. The resin is then washed with washing buffer. The process is repeated for 2-4 times under gentle stirring or in column wash. An additional washing step using a 0.5 M NaCl containing buffer for complete desorption of non-covalently bound proteins can be done. Excess water is removed by filtration. The immobilized antibody can then be characterized in terms of moisture content and specific binding activity.

1. A blood treatment device adapted to remove at least one human complement factor from the blood or blood plasma of a person in need in an extracorporeal blood circuit, wherein the device comprises a matrix configured to immobilize said complement factor.

2. A blood treatment device according to claim 1, wherein the device comprises a matrix configured to immobilize C5.

3. A blood treatment device according to claim 1, wherein the device comprises a matrix configured to immobilize human complement factor 5a (C5a) and/or human complement factor 5b (C5b).

4. A blood treatment device according to claim 2, wherein the matrix is configured to additionally immobilize human complement factor 5a (C5a) and/or human complement factor 5b (C5b).


SEQUENCE LISTING (1 sequence)

SEQ ID NO: 1 - DNA, 741 bp, Mus musculus
gatatccagatgacccagtccccgtcctccctgtccgcctctgtgggcgatagggtcaccatcacctgcggcgccagcgaaaacatctatggcgcgctgaactggtatcaacagaaacccgggaaagctccgaagcttctgatttacggtgcgacgaacctggcagatggagtcccttctcgcttctctggatccggctccggaacggatttcactctgaccatcagcagtctgcagcctgaagacttcgctacgtattactgtcagaacgttttaaatactccgttgactttcggacagggtaccaaggtggaaataaaacgtactggcggtggtggttctggtggcggtggatctggtggtggcggttctcaagtccaactggtgcaatccggcgccgaggtcaagaagccaggggcctcagtcaaagtgtcctgtaaagctagcggctatattttttctaattattggattcaatgggtgcgtcaggcccccgggcagggcctggaatggatgggtgagatcttaccgggctctggtagcaccgaatataccgaaaattttaaagaccgtgttactatgacgcgtgacacttcgactagtacagtatacatggagctctccagcctgcgatcggaggacacggccgtctattattgcgcgcgttatttttttggttctagcccgaattggtattttgatgtttggggtcaaggaaccctggtcactgtctcgagctga